(19) United States
(12) Patent Application Publication
(10) Pub. No.: US 2022/0072217 A1
(43) Pub. Date: Mar. 10, 2022

(54) WOUND DRESSING MATERIAL FOR VISUAL INDICATION OF WOUND PROTEASE ACTIVITY

(71) Applicant: SYSTAGENIX WOUND MANAGEMENT, LIMITED, West Sussex (GB)

(72) Inventors: Alexander WAITE, Keighley (GB); Katherine A. BOURDILLON, Leeds (GB); Carrina WARD, Gargrave (GB)

(21) Appl. No.: 17/415,215

(22) PCT Filed: Dec. 17, 2019
(86) PCT No.: PCT/IB2019/060876

§ 371 Date: Jun. 17, 2021

Related U.S. Application Data
(60) Provisional application No. 62/783,966, filed on Dec. 21, 2018.

(51) Int. Cl. A61M 1/00; A61L 15/42; A61F 13/02; A61L 15/60; A61L 15/22; A61F 13/00

(52) U.S. Cl. CPC A61M 1/95; A61M 2207/00; A61F 13/0263; A61L 15/60; A61L 15/225; A61L 15/425; A61M 1/985; A61F 13/0209; A61F 13/0206; A61F 13/0216; A61F 13/00059; A61F 13/00063; A61L 2300/442; A61L 2300/404; A61L 15/42

(57) ABSTRACT

The present disclosure relates generally to wound dressings and reduced-pressure wound dressing apparatuses that detect the presence of proteases in a wound upon application. The wound dressings and the reduced-pressure wound dressing apparatus of the present technology can be a visual indicator of the presence of proteases in a wound; and a visual indicator of the wounds healing status.

### CROSS-REFERENCE TO RELATED APPLICATIONS

This application claims the benefit of priority to U.S. Patent Application No. 62/783,966, filed on Dec. 21, 2018, the contents of which are incorporated herein in their entirety.

### TECHNICAL FIELD

The present technology relates generally to wound dressings and reduced-pressure wound dressing apparatuses that detect the presence of proteases in a wound upon application, and over time and methods of using the same. Kits for use in practicing the methods are also provided.

### BACKGROUND

The following description of the background of the present technology is provided simply as an aid in understanding the present technology and is not admitted to describe or constitute prior art to the present technology.

Proteases play pivotal roles in normal wound healing processes. In general, different wound-related proteases act on various proteins, including proteins of the extracellular matrix (ECM) and connective tissue. In the normal wound healing process, proteases break down damaged ECM proteins and foreign material so that new tissue can form and wound closure can occur. Excessive wound proteases lead to the breakdown of newly formed ECM and other proteins, and as a result wound healing is impaired due to damage to the ECM and abnormal prolongation of the inflammatory stage. Currently, there is an unmet need for dressings that detect protease levels in a wound upon application, and over time.

### SUMMARY OF THE PRESENT TECHNOLOGY

In one aspect, the present disclosure provides a wound dressing comprising a biopolymer containing a dye that is configured to release at least a portion of the dye in the presence of one or more proteases, an absorbent material that is configured to absorb the dye released by the biopolymer, and a backing layer that is configured to provide visualization of at least some of the dye absorbed by the absorbent material as a result of protease-mediated degradation of the biopolymer.

Additionally or alternatively, in some embodiments, the biopolymer containing the dye is in the form of a film. Additionally or alternatively, in some embodiments, the film containing the biopolymer and the dye comprises perforations. Additionally or alternatively, in some embodiments, the perforations are about 1 mm to about 10 mm. Additionally or alternatively, in some embodiments, the perforations in the film containing the biopolymer and the dye may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

Additionally or alternatively, in some embodiments, the thickness of the biopolymer containing the dye is about 15 μm to about 3 mm. Additionally or alternatively, in some embodiments, the thickness of the biopolymer containing the dye on the wound-interface layer is about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 240 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, or any range including and/or in between any two of the preceding values.

Additionally or alternatively, in some embodiments, the biopolymer containing the dye is composed of one or more of a collagen, a gelatin, an elastin, a fibronectin, or any combination thereof.

Additionally or alternatively, in some embodiments, the biopolymer comprises about 0.01 wt. % to about 10 wt. % dye. Additionally or alternatively, in some embodiments, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values.

Additionally or alternatively, in some embodiments, the dye is selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

Additionally or alternatively, in some embodiments, the one or more proteases are collagenases, stromeolysins, gelatinases, elastases, fibronectinases, membrane-type MMPs, MMP-8, MMP-2 or MMP-9.

Additionally or alternatively, in some embodiments, the solid content of the biopolymer containing the dye comprises about 1% w/v to about 6% w/v. Additionally or alternatively, in some embodiments, the solid content of the biopolymer containing the dye may comprise about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6% w/v, or any range including and/or in between any two of the preceding values.

Additionally or alternatively, in some embodiments, the biopolymer containing the dye comprises at least one plasticizer. Additionally or alternatively, in some embodiments, the biopolymer containing the dye comprises about 0.3% w/v to about 5% w/v of at least one plasticizer. Additionally or alternatively, in some embodiments, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values.

Additionally or alternatively, in some embodiments, the at least one plasticizer is selected from the group consisting of an acetylated monoglyceride, an alkyl citrate, methyl ricinoleate, glycerol, polyvinylpyrrolidone, and any combination thereof. Additionally or alternatively, in some embodiments, the alkyl citrate is triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

Additionally or alternatively, in some embodiments, the absorbent material is substantially white.

Additionally or alternatively, in some embodiments, the absorbent material is a superabsorbent. Additionally or alternatively, in some embodiments, the superabsorbent of the absorbent material comprises about 5 wt. % to about 60 wt. %. Additionally or alternatively, in some embodiments, the superabsorbent of the absorbent material may comprise about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 22 wt. %, about 24 wt. %, about 26 wt. %, about 28 wt. %, about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, or any range including and/or in between any two of the preceding values. Additionally or alternatively, in some embodiments, the superabsorbent of the absorbent material may be sodium polyacrylate.

Additionally or alternatively, in some embodiments, the thickness of the absorbent material is about 15 μm to about 500 μm. Additionally or alternatively, in some embodiments, the thickness of the absorbent material may be about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, or any range including and/or in between any two of the preceding values.

Additionally or alternatively, in some embodiments, the backing layer is transparent or semi-transparent.

Additionally or alternatively, in some embodiments, the backing layer may be selected from the group consisting of polyurethane, polyalkoxy alkyl acrylate, polyalkoxy alkyl methacrylates, and any combination thereof.

Additionally or alternatively, in some embodiments, the thickness of the backing layer is about 10 μm to about 1000 μm, about 30 μm to about 60 μm. Additionally or alternatively, in some embodiments, the thickness of the backing layer may be about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, about 510 μm, about 520 μm, about 530 μm, about 540 μm, about 550 μm, about 560 μm, about 570 μm, about 580 μm, about 590 μm, about 600 μm, about 610 μm, about 620 μm, about 630 μm, about 640 μm, about 650 μm, about 660 μm, about 670 μm, about 680 μm, about 690 μm, about 700 μm, about 710 μm, about 720 μm, about 730 μm, about 740 μm, about 750 μm, about 760 μm, about 770 μm, about 780 μm, about 790 μm, about 800 μm, about 810 μm, about 820 μm, about 830 μm, about 840 μm, about 850 μm, about 860 μm, about 870 μm, about 880 μm, about 890 μm, about 900 μm, about 910 μm, about 920 μm, about 930 μm, about 940 μm, about 950 μm, about 960 μm, about 970 μm, about 980 μm, about 990 μm, about 1000 μm, or any range including and/or in between any two of the preceding values.

Additionally or alternatively, in some embodiments, the backing layer comprises a moisture vapor transmission rate (MVTR) of about 300 g/m²/24 hrs to about 20,000 g/m²/24 hrs, or about 500 g/m²/24 hrs to about 2000 g/m²/24 hrs. Additionally or alternatively, in some embodiments, the backing layer may comprise a MVTR of about 300 g/m²/24 hrs, about 350 g/m²/24 hrs, about 400 g/m²/24 hrs, about 450 g/m²/24 hrs, about 500 g/m²/24 hrs, about 550 g/m²/24 hrs, about 600 g/m²/24 hrs, about 650 g/m²/24 hrs, about 700 g/m²/24 hrs, about 750 g/m²/24 hrs, about 800 g/m²/24 hrs, about 850 g/m²/24 hrs, about 900 g/m²/24 hrs, about 950 g/m²/24 hrs, about 1000 g/m²/24 hrs, about 1100 g/m²/24 hrs, about 1200 g/m²/24 hrs, about 1300 g/m²/24 hrs, about 1400 g/m²/24 hrs, about 1500 g/m²/24 hrs, about 1600 g/m²/24 hrs, about 1700 g/m²/24 hrs, about 1800 g/m²/24 hrs, about 1900 g/m²/24 hrs, about 2000 g/m²/24 hrs, about 2200 g/m²/24 hrs, about 2400 g/m²/24 hrs, about 2600 g/m²/24 hrs, about 2800 g/m²/24 hrs, about 3000 g/m²/24 hrs, about 3200 g/m²/24 hrs, about 3400 g/m²/24 hrs, about 3600 g/m²/24 hrs, about 3800 g/m²/24 hrs, about 4000 g/m²/24 hrs, about 4200 g/m²/24 hrs, about 4400 g/m²/24 hrs, about 4600 g/m²/24 hrs, about 4800 g/m²/24 hrs, about 5000 g/m²/24 hrs, about 5200 g/m²/24 hrs, about 5400 g/m²/24 hrs, about 5600 g/m²/24 hrs, about 5800 g/m²/24 hrs, about 6000 g/m²/24 hrs, about 6200 g/m²/24 hrs, about 6400 g/m²/24 hrs, about 6600 g/m²/24 hrs, about 6800 g/m²/24 hrs, about 7000 g/m²/24 hrs, about 7200 g/m²/24 hrs, about 7400 g/m²/24 hrs, about 7600 g/m²/24 hrs, about 7800 g/m²/24 hrs, about 8000 g/m²/24 hrs, about 8200 g/m²/24 hrs, about 8400 g/m²/24 hrs, about 8600 g/m²/24 hrs, about 8800 g/m²/24 hrs, about 9000 g/m²/24 hrs, about 9200 g/m²/24 hrs, about 9400 g/m²/24 hrs, about 9600 g/m²/24 hrs, about 9800 g/m²/24 hrs, about 10000 g/m²/24 hrs, about 10500 g/m²/24 hrs, about 11000 g/m²/24 hrs, about 11500 g/m²/24 hrs, about 12000 g/m²/24 hrs, about 12500 g/m²/24 hrs, about 13000 g/m²/24 hrs, about 13500 g/m²/24 hrs, about 14000 g/m²/24 hrs, about 14500 g/m²/24 hrs, about 15000 g/m²/24 hrs, about 15500 g/m²/24 hrs, about 16000 g/m²/24 hrs, about 16500 g/m²/24 hrs, about 17000 g/m²/24 hrs, about 17500 g/m²/24 hrs, about 18000 g/m²/24 hrs, about 18500 g/m²/24 hrs, about 19000 g/m²/24 hrs, about 19500 g/m²/24 hrs, about 20000 g/m²/24 hrs, or any range including and/or in between any two of the preceding values.

Additionally or alternatively, in some embodiments, the wound dressing further comprises a wound-interface layer.

Additionally or alternatively, in some embodiments, the wound-interface layer is an absorbent foam.

Additionally or alternatively, in some embodiments, the absorbent foam of the wound-interface layer is one or more of thermoplastic elastomers, GranuFoam®, Supracor®, Grey Foam, Zotefoam, hydropolymer polyurethane foam, or any combination thereof. Additionally or alternatively, in some embodiments, the thermoplastic elastomers are selected from the group consisting of styrene ethylene butylene styrene (SEBS) copolymers and thermoplastic polyurethane (TPU).

Additionally or alternatively, in some embodiments, the thickness of the wound-interface layer is about 15 μm to about 500 μm. Additionally or alternatively, in some embodiments, the thickness of the wound-interface layer may be about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, or any range including and/or in between any two of the preceding values.

Additionally or alternatively, in some embodiments, the wound-interface layer comprises about 0.001 wt. % to about 5 wt. % of an antimicrobial agent. Additionally or alternatively, in some embodiments, the wound-interface layer may comprise about 0.001 wt. % to about 5 wt. % of an antimicrobial agent. Additionally or alternatively, in some embodiments the antimicrobial agent of the wound-interface layer may comprise about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, or any range including and/or in between any two of the preceding values.

Additionally or alternatively, in some embodiments, the antimicrobial agent is selected from the group consisting of tetracycline, penicillins, terramycins, erythromycin, bacitracin, neomycin, polymycin B, mupirocin, clindamycin, colloidal silver, silver salts, silver sulfadiazine, polyhexanide, chlorhexidine, povidone iodine, triclosan, sucralfate, quaternary ammonium salts, and any combination thereof.

Additionally or alternatively, in some embodiments, the wound-interface layer comprises perforations. Additionally or alternatively, in some embodiments, the perforations are about 1 mm to about 10 mm in diameter. Additionally or alternatively, in some embodiments, the perforations of the wound-interface layer may be about 1 mm to about 10 mm in diameter. Thus, the perforations in the wound-interface layer may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

In one aspect, the present disclosure provides a reduced-pressure wound dressing apparatus comprising a wound-interface layer, a biopolymer containing a dye that is configured to release at least a portion of the dye in the presence of one or more proteases, a drape comprising a pressure-sensitive adhesive in peripheral areas for sealing a wound tissue site, a canister for collecting fluids, wherein the canister is configured to be connected to the drape through a first tube connection, and a vacuum for applying negative pressure to said reduced-pressure wound dressing apparatus, wherein the vacuum is configured to be connected to the canister through a second tube connection.

Additionally or alternatively, in some embodiments, the wound-interface layer is an absorbent foam.

Additionally or alternatively, in some embodiments, the absorbent foam of the wound-interface layer is one or more of thermoplastic elastomers, GranuFoam®, Supracor®, Grey Foam, Zotefoam, hydropolymer polyurethane foam, or any combination thereof.

Additionally or alternatively, in some embodiments, the thermoplastic elastomers are selected from the group consisting of styrene ethylene butylene styrene (SEBS) copolymers and thermoplastic polyurethane (TPU).

Additionally or alternatively, in some embodiments, the wound-interface layer comprises a firmness factor (FF) of about 1 to about 5. Additionally or alternatively, in some embodiments, the wound-interface layer may comprise a firmness factor (FF) of about 1, about 1.1, about 1.2, about 1.3, about 1.4, about 1.5, about 1.6, about 1.7, about 1.8, about 1.9, about 2, about 2.2, about 2.4, about 2.6, about 2.8, about 3, about 3.2, about 3.4, about 3.6, about 3.8, about 4, about 4.2, about 4.4, about 4.6, about 4.8, about 5, or any range including and/or in between any two of the preceding values.

Additionally or alternatively, in some embodiments, the wound-interface layer comprises perforations. Additionally or alternatively, in some embodiments, the perforations are about 1 mm to about 10 mm in diameter. Additionally or alternatively, in some embodiments, the perforations in the wound-interface layer may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

Additionally or alternatively, in some embodiments, the wound-interface layer comprises about 0.001 wt. % to about 5 wt. % of an antimicrobial agent. Additionally or alternatively, in some embodiments the antimicrobial agent of the wound-interface layer may comprise about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, or any range including and/or in between any two of the preceding values.

Additionally or alternatively, in some embodiments, the antimicrobial agent is selected from the group consisting of tetracycline, penicillins, terramycins, erythromycin, bacitracin, neomycin, polymycin B, mupirocin, clindamycin, colloidal silver, silver salts, silver sulfadiazine, polyhexanide, chlorhexidine, povidone iodine, triclosan, sucralfate, quaternary ammonium salts, and any combination thereof.

Additionally or alternatively, in some embodiments, the biopolymer containing the dye is composed of one or more of a collagen, a gelatin, an elastin, a fibronectin, or any combination thereof.

Additionally or alternatively, in some embodiments, the biopolymer comprises about 0.01 wt. % to about 10 wt. % dye. Additionally or alternatively, in some embodiments, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values.

Additionally or alternatively, in some embodiments, the dye is selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

Additionally or alternatively, in some embodiments, the one or more proteases are collagenases, stromeolysins, gelatinases, elastases, fibronectinases, membrane-type MMPs, MMP-8, MMP-2 or MMP-9.

Additionally or alternatively, in some embodiments, the biopolymer containing the dye is applied and dehydrated onto the wound-interface layer. Additionally or alternatively, in some embodiments, the thickness of the biopolymer containing the dye on the wound-interface layer is about 15 μm to about 3 mm. Additionally or alternatively, in some embodiments, the thickness of the biopolymer containing the dye on the wound-interface layer is about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 240 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, or any range including and/or in between any two of the preceding values.

Additionally or alternatively, in some embodiments, the solid content of the biopolymer containing the dye comprises about 1% w/v to about 6% w/v. Additionally or alternatively, in some embodiments, the solid content of the biopolymer containing the dye may comprise about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6% w/v, or any range including and/or in between any two of the preceding values.

Additionally or alternatively, in some embodiments, the biopolymer containing the dye comprises at least one plasticizer. Additionally or alternatively, in some embodiments, the biopolymer containing the dye may comprise about 0.3% w/v to about 5% w/v of at least one plasticizer. Additionally or alternatively, in some embodiments, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values.

Additionally or alternatively, in some embodiments, the at least one plasticizer is selected from the group consisting of an acetylated monoglyceride, an alkyl citrate, methyl ricinoleate, glycerol, polyvinylpyrrolidone, and any combination thereof. Additionally or alternatively, in some embodiments, the alkyl citrate is triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

Additionally or alternatively, in some embodiments, the drape comprises a polyurethane film or an elastomeric film. Additionally or alternatively, in some embodiments, the elastomeric film is natural rubber, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, ethylene vinyl acetate (EVA) film, co-polyester, silicone, or any combination thereof.

Additionally or alternatively, in some embodiments, the thickness of the drape is about 30 μm to about 100 μm. Additionally or alternatively, in some embodiments, the thickness of the drape may be about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, or any range including and/or in between any two of the preceding values.

Additionally or alternatively, in some embodiments, the drape comprises a moisture vapor transmission rate (MVTR) of about 300 g/m²/24 hrs to about 20,000 g/m²/24 hrs, or about 500 g/m²/24 hrs to about 2000 g/m²/24 hrs. Additionally or alternatively, in some embodiments, the drape may comprise a MVTR of about 300 g/m²/24 hrs, about 350 g/m²/24 hrs, about 400 g/m²/24 hrs, about 450 g/m²/24 hrs, about 500 g/m²/24 hrs, about 550 g/m²/24 hrs, about 600 g/m²/24 hrs, about 650 g/m²/24 hrs, about 700 g/m²/24 hrs, about 750 g/m²/24 hrs, about 800 g/m²/24 hrs, about 850 g/m²/24 hrs, about 900 g/m²/24 hrs, about 950 g/m²/24 hrs, about 1000 g/m²/24 hrs, about 1100 g/m²/24 hrs, about 1200 g/m²/24 hrs, about 1300 g/m²/24 hrs, about 1400 g/m²/24 hrs, about 1500 g/m²/24 hrs, about 1600 g/m²/24 hrs, about 1700 g/m²/24 hrs, about 1800 g/m²/24 hrs, about 1900 g/m²/24 hrs, about 2000 g/m²/24 hrs, about 2200 g/m²/24 hrs, about 2400 g/m²/24 hrs, about 2600 g/m²/24 hrs, about 2800 g/m²/24 hrs, about 3000 g/m²/24 hrs, about 3200 g/m²/24 hrs, about 3400 g/m²/24 hrs, about 3600 g/m²/24 hrs, about 3800 g/m²/24 hrs, about 4000 g/m²/24 hrs, about 4200 g/m²/24 hrs, about 4400 g/m²/24 hrs, about 4600 g/m²/24 hrs, about 4800 g/m²/24 hrs, about 5000 g/m²/24 hrs, about 5200 g/m²/24 hrs, about 5400 g/m²/24 hrs, about 5600 g/m²/24 hrs, about 5800 g/m²/24 hrs, about 6000 g/m²/24 hrs, about 6200 g/m²/24 hrs, about 6400 g/m²/24 hrs, about 6600 g/m²/24 hrs, about 6800 g/m²/24 hrs, about 7000 g/m²/24 hrs, about 7200 g/m²/24 hrs, about 7400 g/m²/24 hrs, about 7600 g/m²/24 hrs, about 7800 g/m²/24 hrs, about 8000 g/m²/24 hrs, about 8200 g/m²/24 hrs, about 8400 g/m²/24 hrs, about 8600 g/m²/24 hrs, about 8800 g/m²/24 hrs, about 9000 g/m²/24 hrs, about 9200 g/m²/24 hrs, about 9400 g/m²/24 hrs, about 9600 g/m²/24 hrs, about 9800 g/m²/24 hrs, about 10000 g/m²/24 hrs, about 10500 g/m²/24 hrs, about 11000 g/m²/24 hrs, about 11500 g/m²/24 hrs, about 12000 g/m²/24 hrs, about 12500 g/m²/24 hrs, about 13000 g/m²/24 hrs, about 13500 g/m²/24 hrs, about 14000 g/m²/24 hrs, about 14500 g/m²/24 hrs, about 15000 g/m²/24 hrs, about 15500 g/m²/24 hrs, about 16000 g/m²/24 hrs, about 16500 g/m²/24 hrs, about 17000 g/m²/24 hrs, about 17500 g/m²/24 hrs, about 18000 g/m²/24 hrs, about 18500 g/m²/24 hrs, about 19000 g/m²/24 hrs, about 19500 g/m²/24 hrs, about 20000 g/m²/24 hrs, or any range including and/or in between any two of the preceding values.

Additionally or alternatively, in some embodiments, the first tube connection and the second tube connection may independently be selected from the group consisting of polyvinyl chloride, polyethylene, polypropylene, and any combination thereof.

Additionally or alternatively, in some embodiments, the vacuum is used to apply negative pressure to a wound. Additionally or alternatively, in some embodiments, the negative pressure applied to a wound may be about −5 mm Hg to about −500 mm Hg, or about −75 mm Hg to about −300 mm Hg. Additionally or alternatively, in some embodiments, the negative pressure applied to a wound may be about −5 mm Hg, about −6 mm Hg, about −7 mm Hg, about −8 mm Hg, about −9 mm Hg, about −10 mm Hg, about −11 mm Hg, about −12 mm Hg, about −13 mm Hg, about −14 mm Hg, about −15 mm Hg, about −16 mm Hg, about −17 mm Hg, about −18 mm Hg, about −19 mm Hg, about −20 mm Hg, about −22 mm Hg, about −24 mm Hg, about −26 mm Hg, about −28 mm Hg, about −30 mm Hg, about −32 mm Hg, about −34 mm Hg, about −36 mm Hg, about −38 mm Hg, about −40 mm Hg, about −42 mm Hg, about −44 mm Hg, about −46 mm Hg, about −48 mm Hg, about −50 mm Hg, about −52 mm Hg, about −54 mm Hg, about −56 mm Hg, about −58 mm Hg, about −60 mm Hg, about −62 mm Hg, about −64 mm Hg, about −66 mm Hg, about −68 mm Hg, about −70 mm Hg, about −72 mm Hg, about −74 mm Hg, about −76 mm Hg, about −78 mm Hg, about −80 mm Hg, about −82 mm Hg, about −84 mm Hg, about −86 mm Hg, about −88 mm Hg, about −90 mm Hg, about −92 mm Hg, about −94 mm Hg, about −96 mm Hg, about −98 mm Hg, about −100 mm Hg, about −110 mm Hg, about −120 mm Hg, about −130 mm Hg, about −140 mm Hg, about −150 mm Hg, about −160 mm Hg, about −170 mm Hg, about −180 mm Hg, about −190 mm Hg, about −200 mm Hg, about −220 mm Hg, about −240 mm Hg, about −260 mm Hg, about −280 mm Hg, about −300 mm Hg, about −320 mm Hg, about −340 mm Hg, about −360 mm Hg, about −380 mm Hg, about −400 mm Hg, about −420 mm Hg, about −440 mm Hg, about −460 mm Hg, about −480 mm Hg, about −500 mm Hg, or any range including and/or in between any two of these values.

In one aspect, the present disclosure provides a method for detecting protease activity levels in a wound in a subject in need thereof, the method comprising administering a wound dressing of any embodiment disclosed herein, and detecting a colorimetric signal in the absorbent material of the wound dressing, wherein the presence of the colorimetric signal indicates protease activity in the wound.

In one aspect, the present disclosure provides a method for detecting delays in wound healing in a subject in need thereof, the method comprising administering to the wound a wound dressing of any embodiment disclosed herein, determining a first protease activity level by detecting a first colorimetric signal in the absorbent material of the wound dressing when the wound dressing is administered to the subject, and determining a second protease activity level by detecting a second colorimetric signal in the absorbent material of the wound dressing about 10 minutes to about 7 days after the wound dressing is administered to the subject; wherein wound healing is delayed when the second protease activity level is greater compared to the first protease activity level.

In one aspect, the present disclosure provides a method for detecting delays in wound healing in a subject in need thereof, the method comprising administering to the wound a wound dressing of any embodiment disclosed herein, detecting a colorimetric signal in the absorbent material of the wound dressing, wherein the colorimetric change indicates elevated protease activity levels, and determining a protease activity level compared to a pre-determined reference level.

In one aspect, the present disclosure provides a method for detecting protease activity levels in a wound in a subject in need thereof, the method comprising contacting the wound with the wound-interface layer of the reduced-pressure wound dressing apparatus of any embodiment disclosed herein, applying negative pressure using the vacuum of the reduced-pressure wound dressing apparatus, collecting wound exudate via the first tube connection and/or canister of the reduced-pressure wound dressing apparatus, and detecting a colorimetric signal in the collected wound exudate, wherein detection of the colorimetric signal indicates protease activity in the wound.

In one aspect, the present disclosure provides a method for detecting delays in wound healing in a subject in need thereof, the method comprising contacting the wound with the wound-interface layer of the reduced-pressure wound dressing apparatus of any embodiment disclosed herein, applying negative pressure via the vacuum of the reduced-pressure wound dressing apparatus, collecting wound exudate via the first tube connection and/or canister of the reduced-pressure wound dressing apparatus, detecting a first colorimetric signal in the wound exudate at a first time period, detecting a second colorimetric signal in the wound exudate at a second time period, and detecting a delay in wound healing when the second colorimetric signal is greater than the first colorimetric signal.

In one aspect, the present disclosure provides a method for making a wound dressing, the method comprising providing a biopolymer containing a dye that is configured to release at least a portion of the dye in the presence of one or more proteases, an absorbent material configured to absorb the dye released by the biopolymer, a backing layer configured to provide visibility to a user of at least some of the dye absorbed by the absorbent material, and combining the biopolymer containing the dye, the absorbent material and the backing layer to make the wound dressing.

In one aspect, the present disclosure provides a method for making a reduced-pressure wound dressing apparatus, the method comprising, providing a biopolymer containing a dye and configured to release at least a portion of the dye when in the presence of one or more proteases, a drape comprising a pressure-sensitive adhesive in peripheral areas for sealing a wound tissue site, a canister for collecting fluids, wherein the canister is configured to be connected to the drape through a first tube connection, a vacuum for applying negative pressure to said reduced-pressure wound dressing apparatus, wherein the vacuum is configured to be connected to the canister through a second tube connection, and combining the biopolymer containing the dye, the drape, the canister, and the vacuum to make the reduced-pressure wound dressing apparatus.

Also provided herein are kits comprising the wound dressings of any embodiment disclosed herein and instructions for use.

Also provided herein are kits comprising the reduced-pressure wound dressing apparatuses of any embodiment disclosed herein and instructions for use.

### BRIEF DESCRIPTION OF THE DRAWINGS

FIG. 1 shows a diagrammatic representation of an embodiment of a wound dressing of the present technology.

FIG. 2 shows a diagrammatic representation of an embodiment of a reduced-pressure wound dressing apparatus of the present technology.

### DETAILED DESCRIPTION

It is to be appreciated that certain aspects, modes, embodiments, variations and features of the present methods are described below in various levels of detail in order to provide a substantial understanding of the present technology.

Proteases play pivotal roles in normal wound healing processes. In general, different wound-related proteases act on various proteins, including proteins of the extracellular matrix (ECM) and connective tissue. In the normal wound healing process, proteases break down damaged ECM proteins and foreign material so that new tissue can form and wound closure can occur. Excessive wound proteases lead to the breakdown of newly formed ECM and other proteins, and as a result wound healing is impaired due to damage to the ECM and abnormal prolongation of the inflammatory stage.

The present disclosure is directed to wound dressings and reduced-pressure wound dressing apparatuses that include a biopolymer containing a dye, which can be configured to release the dye in the presence of proteases found in the wound exudate. Thus, the wound dressings and reduced-pressure wound dressing apparatuses of the present technology will advantageously allow for the monitoring of protease activity levels in a wound in a subject in need thereof.

Thus, in one aspect, a wound dressing is provided that includes a biopolymer containing a dye that is configured to release at least a portion of the dye in the presence of one or more proteases, an absorbent material configured to absorb the dye released by the biopolymer, and a backing layer configured to provide visualization of at least some of the dye absorbed by the absorbent material as a result of protease-mediated degradation of the biopolymer.

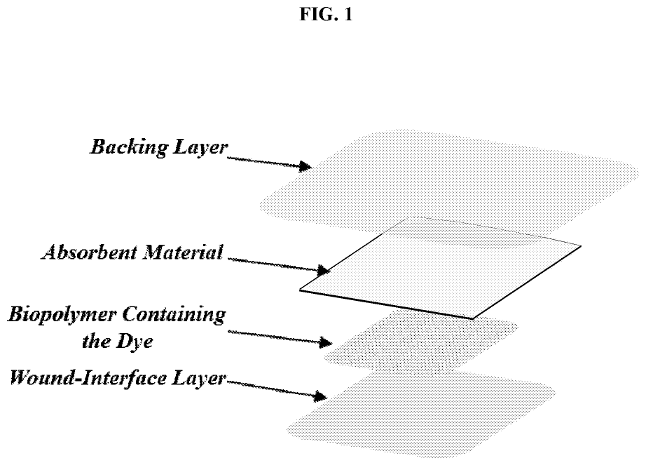

FIG. 1 provides a non-limiting representative illustration of an embodiment of each layer of the wound dressing of the present technology.

In another aspect, a reduced-pressure wound dressing apparatus is provided that includes a wound-interface layer, a biopolymer containing a dye that is configured to release at least a portion of the dye in the presence of one or more proteases, a drape comprising a pressure-sensitive adhesive in peripheral areas for sealing a wound tissue site, a canister for collecting fluids, wherein the canister is configured to be connected to the drape through a first tube connection, and a vacuum for applying negative pressure to said reduced-pressure wound dressing apparatus, wherein the vacuum is configured to be connected to the canister through a second tube connection.

“Negative pressure” may refer to a pressure less than a local ambient pressure, such as the ambient pressure in a local environment that is external to a sealed therapeutic environment provided by a dressing. Additionally or alternatively, in some embodiments, the local ambient pressure may also be the atmospheric pressure proximate to a wound site. Additionally or alternatively, in some embodiments, the local ambient pressure may also be less than a hydrostatic pressure associated with a wound site. Additionally or alternatively, in some embodiments, NPWT may provide a number of benefits, including, but not limited to, migration of epithelial and subcutaneous tissues, improved blood flow, and micro-deformation of tissue at a wound site. These benefits may increase development of granulation tissue and reduce healing times. Additionally or alternatively, in some embodiments, a negative pressure applied across a wound, via the reduced-pressure wound dressing apparatus may be effective to induce macrostrain and microstrain at wound site, as well as remove exudates and other fluids from the wound site.

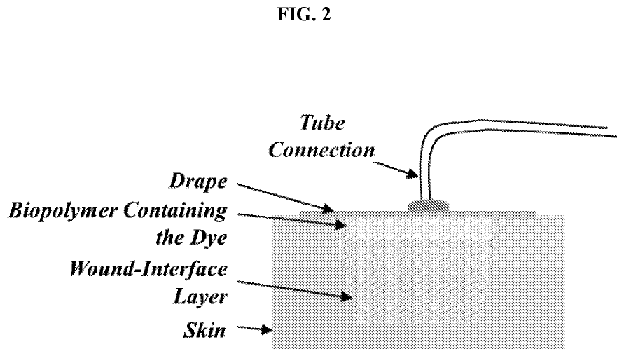

FIG. 2 provides a non-limiting representative illustration of an embodiment of a reduced-pressure wound dressing apparatus of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the “administration” of a wound dressing or a reduced-pressure wound dressing apparatus to a subject includes any route of introducing or delivering to a subject a diagnostic wound dressing composition to perform its intended function. Administration can be carried out by any suitable route, including but not limited to, topical administration. Administration includes self-administration and the administration by another.

As used herein, the terms “contain”, “contains”, or “containing” in the context of describing the elements (especially in the context of the following claims) are to be construed as comprising or including the elements being described herein.

As used herein, the term “effective amount” refers to a quantity sufficient to achieve a desired therapeutic and/or prophylactic effect, e.g., an amount which results in the decrease in a wound described herein or one or more signs or symptoms associated with a wound described herein. In the context of therapeutic or prophylactic applications, the wound dressing or reduced-pressure wound dressing apparatus administered to the subject will vary depending on the composition, the degree, type, and severity of the wound and on the characteristics of the individual.

As used herein, the term “firmness factor” refers to a ratio of the density of a foam in a compressed state to the density of the same foam in an uncompressed state. For example, a firmness factor (FF) of 5 may refer to a foam in a compressed state having a density that is 5 times greater than that of the density of the same foam in an uncompressed state.

As used herein, the terms “individual”, “patient”, or “subject” can be an individual organism, a vertebrate, a mammal, or a human. In some embodiments, the individual, patient or subject is a human.

As used herein, the terms “moisture vapor transmission rate” and “MVTR” will be understood by persons of ordinary skill in the art as a measure of the passage of water vapor through a substance of a given unit area and unit time. The most common international unit for the MVTR is g/m²/day, wherein 1 day=24 hr.

As understood by one of ordinary skill in the art, “molecular weight” (also known as “relative molar mass”) is a dimensionless quantity that can be converted to molar mass by multiplying by 1 gram/mole—for example, collagen with a weight-average molecular weight of 5,000 has a weight-average molar mass of 5,000 g/mol.

As used herein, the term “NPWT” refers to negative pressure wound therapy, which is a type of wound therapy that involves applying negative pressure (relative to atmospheric pressure) to a wound bed to promote wound healing. Typically, a dressing is sealed over a wound site and air is pumped out of the dressing to create negative pressure at the wound site. In some NPWT systems, wound exudate and other fluid is pumped out of the dressing and collected by a canister.

As used herein, the term “solid content” refers to the density of a material and/or film of the wound dressing or reduced-pressure wound dressing apparatus of the present technology, which is its mass per unit volume.

As used herein, the term “substantial” and “substantially” includes total but also less than total. In the context of the wound dressing of the present technology, the absorbent material which absorbs the dye released from the biopolymer is substantially white.

As used herein, the terms “superabsorbent” and “superabsorbent polymer (SAP)” will be understood by persons of ordinary skill in the art as materials which can absorb and retain extremely large amounts of fluids relative to their own mass. A SAP may absorb 300 times its weight (from 30 to 60 times its own volume) and become 99.9% liquid.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are, e.g., alleviated, reduced, cured, or placed in a state of remission.

As used herein, the term “% w/v” refers to the percent of weight of the solution in the total volume of the solution, i.e., the number of grams of solute in 100 mL of solution.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single, or several administrations for the treatment of an acute wound.

### The Wound Dressing of the Present Technology

### The Biopolymer Containing the Dye

The present disclosure provides a wound dressing comprising a biopolymer that contains a dye and is configured to release at least a portion of the dye in the presence of one or more proteases.

In any embodiment disclosed herein, the biopolymer containing the dye comprises a wound-facing side and an environmental-facing side.

In any embodiment disclosed herein, the biopolymer containing the dye may be composed of one or more of a collagen, a gelatin, an elastin, a fibronectin, or any combination thereof.

In any embodiment disclosed herein, the thickness of the biopolymer containing the dye may be about 15 μm to about 3 mm. Additionally or alternatively, in some embodiments, the thickness of the biopolymer containing the dye on the wound-interface layer is about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 240 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, or any range including and/or in between any two of the preceding values.

In any embodiment disclosed herein, the biopolymer containing the dye may be in the form of a film. Additionally or alternatively, in some embodiments, the film containing the biopolymer and the dye may include perforations. Additionally or alternatively, in some embodiments, the perforations in the film containing the biopolymer and the dye may be about 1 mm to about 10 mm. Thus, the perforations in the film containing the biopolymer and the dye may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

In any embodiment disclosed herein, the biopolymer may comprise about 0.01 wt. % to about 10 wt. % dye. Additionally or alternatively, in some embodiments, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values. Additionally or alternatively, in some embodiments, the dye may be selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

In any embodiment disclosed herein, the biopolymer containing the dye in the wound dressing of the present technology is configured to release at least a portion of the dye in the presence of one or more proteases in the wound. The wound dressing of the present technology is suitable for use with a variety of proteases. Typically, the proteases selected for use with the wound dressing of the present technology are associated with wound chronicity and delayed healing. Additionally or alternatively, in some embodiments, the one or more proteases are collagenases, stromeolysins, gelatinases, elastases, fibronectinases, membrane-type MMPs, MMP-8, MMP-2 or MMP-9.

In any embodiment disclosed herein, the solid content of the biopolymer containing the dye may comprise about 1% w/v to about 6% w/v. Additionally or alternatively, in some embodiments, the solid content of the biopolymer containing the dye may comprise about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6% w/v, or any range including and/or in between any two of the preceding values.

In any embodiment disclosed herein, the biopolymer containing the dye may comprise at least one plasticizer. Additionally or alternatively, in some embodiments, the biopolymer containing the dye may comprise about 0.3% w/v to about 5% w/v of at least one plasticizer. Additionally or alternatively, in some embodiments, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values. Exemplary plasticizers include, but are not limited to, an acetylated monoglyceride, an alkyl citrate, methyl ricinoleate, glycerol, polyvinylpyrrolidone, or any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

### The Absorbent Material

The present disclosure provides a wound dressing comprising an absorbent material that is configured to absorb a dye released by a biopolymer.

In any embodiment disclosed herein, the absorbent material comprises a wound-facing side and an environmental-facing side.

In any embodiment disclosed herein, the absorbent material is substantially white. Dye that is released from the biopolymer film in the presence of one or more proteases in the wound exudate is absorbed by the absorbent material. The absorbed dye is clearly visible through the backing layer of the wound dressing of the present technology. The presence and the intensity of the dye on the absorbent material provides a direct indication of the protease activity in the wound, which relates to wound chronicity and delayed wound healing.

In any embodiment disclosed herein, the absorbent material is a superabsorbent. Additionally or alternatively, in some embodiments, the superabsorbent of the absorbent material may comprise about 5 wt. % to about 60 wt. %. Additionally or alternatively, in some embodiments, the superabsorbent of the absorbent material may comprise about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 22 wt. %, about 24 wt. %, about 26 wt. %, about 28 wt. %, about 30 wt. %, about 32 wt. %, about 34 wt. %, about 36 wt. %, about 38 wt. %, about 40 wt. %, about 42 wt. %, about 44 wt. %, about 46 wt. %, about 48 wt. %, about 50 wt. %, about 52 wt. %, about 54 wt. %, about 56 wt. %, about 58 wt. %, about 60 wt. %, or any range including and/or in between any two of the preceding values. Additionally or alternatively, in some embodiments, the superabsorbent of the absorbent material may be sodium polyacrylate.

In any embodiment disclosed herein, the thickness of the absorbent material may be about 15 μm to about 500 μm. Additionally or alternatively, in some embodiments, the thickness of the absorbent material may be about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, or any range including and/or in between any two of the preceding values.

### The Backing Layer

The present disclosure provides a backing layer configured to provide visualization of at least some of the dye absorbed by the absorbent material as a result of protease-mediated degradation of a biopolymer.

In any embodiment disclosed herein, the backing layer comprises a wound-facing side and an environmental-facing side.

In any embodiment disclosed herein, the backing layer may be transparent or semi-transparent. Thus, the backing layer provides the ability to visualize the absorption of the dye into the absorbent layer after it is released from the biopolymer due to elevated protease levels in the wound.

In any embodiment disclosed herein, the backing layer may be composed of a material selected from the group consisting of polyurethane, polyalkoxy alkyl acrylate, polyalkoxy alkyl methacrylates, and any combination thereof. Additionally or alternatively, in some embodiments, the thickness of the backing layer may be about 10 μm to about 1000 μm, about 30 μm to about 60 μm. Additionally or alternatively, in some embodiments, the thickness of the backing layer may be about 10 μm, about 11 μm, about 12 μm, about 13 μm, about 14 μm, about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, about 510 μm, about 520 μm, about 530 μm, about 540 μm, about 550 μm, about 560 μm, about 570 μm, about 580 μm, about 590 μm, about 600 μm, about 610 μm, about 620 μm, about 630 μm, about 640 μm, about 650 μm, about 660 μm, about 670 μm, about 680 μm, about 690 μm, about 700 μm, about 710 μm, about 720 μm, about 730 μm, about 740 μm, about 750 μm, about 760 μm, about 770 μm, about 780 μm, about 790 μm, about 800 μm, about 810 μm, about 820 μm, about 830 μm, about 840 μm, about 850 μm, about 860 μm, about 870 μm, about 880 μm, about 890 μm, about 900 μm, about 910 μm, about 920 μm, about 930 μm, about 940 μm, about 950 μm, about 960 μm, about 970 μm, about 980 μm, about 990 μm, about 1000 μm, or any range including and/or in between any two of the preceding values.

In any embodiment disclosed herein, the backing layer is substantially impermeable to liquid or wound exudate. Additionally or alternatively, in some embodiments, the backing layer is microorganism impermeable. Additionally or alternatively, in some embodiments, the backing layer is semi-permeable to water vapor. In any embodiment disclosed herein, the backing layer may comprise a moisture vapor transmission rate (MVTR) of about 300 g/m²/24 hrs to about 20,000 g/m²/24 hrs, or about 500 g/m²/24 hrs to about 2000 g/m²/24 hrs at 37.5° C. at 50% relative humidity difference as described in ASTM E96-00. Thus, the backing layer may comprise a MVTR of about 300 g/m²/24 hrs, about 350 g/m²/24 hrs, about 400 g/m²/24 hrs, about 450 g/m²/24 hrs, about 500 g/m²/24 hrs, about 550 g/m²/24 hrs, about 600 g/m²/24 hrs, about 650 g/m²/24 hrs, about 700 g/m²/24 hrs, about 750 g/m²/24 hrs, about 800 g/m²/24 hrs, about 850 g/m²/24 hrs, about 900 g/m²/24 hrs, about 950 g/m²/24 hrs, about 1000 g/m²/24 hrs, about 1100 g/m²/24 hrs, about 1200 g/m²/24 hrs, about 1300 g/m²/24 hrs, about 1400 g/m²/24 hrs, about 1500 g/m²/24 hrs, about 1600 g/m²/24 hrs, about 1700 g/m²/24 hrs, about 1800 g/m²/24 hrs, about 1900 g/m²/24 hrs, about 2000 g/m²/24 hrs, about 2200 g/m²/24 hrs, about 2400 g/m²/24 hrs, about 2600 g/m²/24 hrs, about 2800 g/m²/24 hrs, about 3000 g/m²/24 hrs, about 3200 g/m²/24 hrs, about 3400 g/m²/24 hrs, about 3600 g/m²/24 hrs, about 3800 g/m²/24 hrs, about 4000 g/m²/24 hrs, about 4200 g/m²/24 hrs, about 4400 g/m²/24 hrs, about 4600 g/m²/24 hrs, about 4800 g/m²/24 hrs, about 5000 g/m²/24 hrs, about 5200 g/m²/24 hrs, about 5400 g/m²/24 hrs, about 5600 g/m²/24 hrs, about 5800 g/m²/24 hrs, about 6000 g/m²/24 hrs, about 6200 g/m²/24 hrs, about 6400 g/m²/24 hrs, about 6600 g/m²/24 hrs, about 6800 g/m²/24 hrs, about 7000 g/m²/24 hrs, about 7200 g/m²/24 hrs, about 7400 g/m²/24 hrs, about 7600 g/m²/24 hrs, about 7800 g/m²/24 hrs, about 8000 g/m²/24 hrs, about 8200 g/m²/24 hrs, about 8400 g/m²/24 hrs, about 8600 g/m²/24 hrs, about 8800 g/m²/24 hrs, about 9000 g/m²/24 hrs, about 9200 g/m²/24 hrs, about 9400 g/m²/24 hrs, about 9600 g/m²/24 hrs, about 9800 g/m²/24 hrs, about 10000 g/m²/24 hrs, about 10500 g/m²/24 hrs, about 11000 g/m²/24 hrs, about 11500 g/m²/24 hrs, about 12000 g/m²/24 hrs, about 12500 g/m²/24 hrs, about 13000 g/m²/24 hrs, about 13500 g/m²/24 hrs, about 14000 g/m²/24 hrs, about 14500 g/m²/24 hrs, about 15000 g/m²/24 hrs, about 15500 g/m²/24 hrs, about 16000 g/m²/24 hrs, about 16500 g/m²/24 hrs, about 17000 g/m²/24 hrs, about 17500 g/m²/24 hrs, about 18000 g/m²/24 hrs, about 18500 g/m²/24 hrs, about 19000 g/m²/24 hrs, about 19500 g/m²/24 hrs, about 20000 g/m²/24 hrs, or any range including and/or in between any two of the preceding values. Such moisture vapor transmission rates allow the wound under the wound dressing to heal under moist conditions without causing the skin surrounding the wound to macerate.

In any embodiment disclosed herein, the backing layer may extend over each of the biopolymer containing the dye and the absorbent material, such that a marginal region of width about 1 mm to about 50 mm, or about 5 mm to about 20 mm extends around wound dressing. In such cases, the wound-facing side of the extended region of the backing layer is suitably coated with a pressure sensitive medical grade adhesive in at least its marginal region. Additionally or alternatively, the marginal region of the backing layer may comprise a width of about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 22 mm, about 24 mm, about 26 mm, about 28 mm, about 30 mm, about 32 mm, about 34 mm, about 36 mm, about 38 mm, about 40 mm, about 42 mm, about 44 mm, about 46 mm, about 48 mm, about 50 mm, or any range including and/or in between any two of the preceding values.

### The Wound-Interface Layer

In any embodiment disclosed herein, the wound dressing may further comprise a wound-interface layer.

In any embodiment disclosed herein, the wound-interface layer comprises a wound-facing side and an environmental-facing side.

In any embodiment disclosed herein, the wound-interface layer comprises an absorbent foam. Additionally or alternatively, in some embodiments, the absorbent foam of the wound-interface layer is one or more of thermoplastic elastomers, GranuFoam®, Supracor®, Grey Foam, Zotefoam, hydropolymer polyurethane foam, or any combination thereof. Additionally or alternatively, in some embodiments, the one or more thermoplastic elastomers are selected from the group consisting of styrene ethylene butylene styrene (SEBS) copolymers and thermoplastic polyurethane (TPU).

In any embodiment disclosed herein, the thickness of the wound-interface layer may be about 15 μm to about 500 μm. Additionally or alternatively, in some embodiments, the thickness of the wound-interface layer may be about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, about 500 μm, or any range including and/or in between any two of the preceding values.

In any embodiment disclosed herein, the wound-interface layer may comprise an antimicrobial agent. Additionally or alternatively, in some embodiments, the wound-interface layer may comprise about 0.001 wt. % to about 5 wt. % of an antimicrobial agent. Additionally or alternatively, in some embodiments the antimicrobial agent of the wound-interface layer may comprise about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, or any range including and/or in between any two of the preceding values. Additionally or alternatively, in some embodiments the antimicrobial agent of the wound-interface layer is selected from the group consisting of tetracycline, penicillins, terramycins, erythromycin, bacitracin, neomycin, polymycin B, mupirocin, clindamycin, colloidal silver, silver salts, silver sulfadiazine, chlorhexidine, povidone iodine, triclosan, sucralfate, quaternary ammonium salts, and any combination thereof.

In any embodiment disclosed herein, the wound-interface layer may comprise perforations. Additionally or alternatively, in some embodiments, the perforations of the wound-interface layer may be about 1 mm to about 10 mm in diameter. Thus, the perforations in the wound-interface layer may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

### The Wound Dressing

The present disclosure provides a wound dressing composition comprising a biopolymer containing a dye, an absorbent material configured to absorb the dye released by the biopolymer, and a backing layer configured to provide visualization of at least some of the dye absorbed by the absorbent material as a result of protease-mediated degradation of the biopolymer, and optionally a wound-interface layer, wherein each of the biopolymer containing the dye, the absorbent material, the backing layer, and optionally the wound-interface layer independently comprise a wound-facing side and an environmental-facing side. The wound dressing composition may comprise any biopolymer containing a dye described herein, any absorbent material described herein, and/or any backing layer described herein.

In any embodiment of the wound dressing disclosed herein, the wound-facing side of the backing layer is adjoined with the environmental-facing side of the absorbent material, and wherein the wound-facing side of the absorbent material is adjoined with the environmental-facing side of the biopolymer containing the dye.

In any embodiment of the wound dressing disclosed herein, the wound-facing side of the backing layer is adjoined with the environmental-facing side of the absorbent material, and wherein the wound-facing side of the absorbent material is adjoined with the environmental-facing side of the biopolymer containing the dye, and wherein the wound-facing side of the biopolymer containing the dye is adjoined with the environmental-facing side of the wound-interface layer.

In any embodiment disclosed herein, the wound dressing of the present technology may be sterile and packaged in a microorganism-impermeable container.

### Detection Methods of the Present Technology

In one aspect, the present disclosure provides a method for detecting protease activity levels in wound in a subject in need thereof, wherein the method comprises administering to the wound a wound dressing of any embodiment described herein, and detecting a colorimetric signal in the absorbent material of the wound dressing, wherein the presence of the colorimetric signal indicates protease activity in the wound. Additionally or alternatively, in some embodiments, the wound may be an acute wound or a chronic wound. Additionally or alternatively, in some embodiments, the wound is an acute wound selected from the group consisting of surgical wounds, trauma wounds, burns, graft sites, and donor sites. Additionally or alternatively, the wound is a chronic wound selected from the group consisting of infectious wounds, venous ulcers, arterial ulcers, ischemic ulcers, decubitis ulcers, and diabetic ulcers.

Any method known to those in the art for administering a wound dressing to an acute wound or a chronic wound disclosed herein may be employed. Suitable methods include in vitro or in vivo methods. In vivo methods typically include the administration of one or more wound dressing compositions to a subject in need thereof, suitably a human.

In another aspect, the present disclosure provides a method for detecting delays in wound healing in a subject in need thereof, wherein the method comprises administering to the wound a wound dressing of any embodiment described herein, determining a first protease activity level by detecting a first colorimetric signal in the absorbent material of the wound dressing when the wound dressing is administered to the subject, and determining a second protease activity level by detecting a second colorimetric signal in the absorbent material of the wound dressing at least 10 minutes, 11 minutes, 12 minutes, 13 minutes, 14 minutes, 15 minutes, 16 minutes, 17 minutes, 18 minutes, 19 minutes, 20 minutes, 22 minutes, 24 minutes, 26 minutes, 28 minutes, 30 minutes, 32 minutes, 34 minutes, 36 minutes, 38 minutes, 40 minutes, 42 minutes, 44 minutes, 46 minutes, 48 minutes, 50 minutes, 52 minutes, 54 minutes, 56 minutes, 58 minutes, 60 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 2 days, 3 days, 4 days, 5 days, 6 days, 7 days, or any range including and/or in between any two of the preceding values, after the wound dressing is administered to the subject, wherein wound healing is delayed when the second protease activity level is greater than the first protease activity level.

In another aspect, the present disclosure provides a method for detecting delays in wound healing in a subject in need thereof, wherein the method comprises administering to the wound a wound dressing of any embodiment disclosed herein, detecting a colorimetric signal in the absorbent material of the wound dressing, wherein the colorimetric change indicates elevated protease activity levels, and determining a protease activity level compared to a pre-determined reference level. Elevated wound protease levels lead to impaired wound healing. Additionally or alternatively, in some embodiments, a pre-determined reference level can be set by a person of ordinary skill in the art at 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 22 minutes, 24 minutes, 26 minutes, 28 minutes, 30 minutes, 32 minutes, 34 minutes, 36 minutes, 38 minutes, 40 minutes, 42 minutes, 44 minutes, 46 minutes, 48 minutes, 50 minutes, 52 minutes, 54 minutes, 56 minutes, 58 minutes, 60 minutes, or any range including and/or in between any two of the preceding values, after the wound dressing of any embodiment herein is administered to a wound.

In any embodiment of the methods of the present technology, a wound dressing disclosed herein is administered to a subject in need thereof. Without wishing to be bound by theory, it is believed that, the wound exudate of the subject may vary in viscosity and quantity, thus affecting the appropriate temporal window for administering the wound dressings disclosed herein. Additionally or alternatively, in some embodiments, the diffusion rate of the wound exudate may vary depending on the structure of the wound dressing composition disclosed herein. Additionally or alternatively, in some embodiments, the wound dressings are administered for about 1 minute or more. Additionally or alternatively, in some embodiments, the wound dressings are administered for about 1 minute, about 2 minutes, about 3 minutes, about 4 minutes, about 5 minutes, about 6 minutes, about 7 minutes, about 8 minutes, about 9 minutes, about 10 minutes, or more. Additionally or alternatively, in some embodiments, the wound dressings are administered for about 10 minutes, about 11 minutes, about 12 minutes, about 13 minutes, about 14 minutes, about 15 minutes, about 16 minutes, about 17 minutes, about 18 minutes, about 19 minutes, about 20 minutes, or more. Additionally or alternatively, in some embodiments, the wound dressings are administered for about 20 minutes, about 22 minutes, about 24 minutes, about 26 minutes, about 28 minutes, about 30 minutes, about 32 minutes, about 34 minutes, about 36 minutes, about 38 minutes, about 40 minutes, about 42 minutes, about 44 minutes, about 46 minutes, about 48 minutes, about 50 minutes, about 52 minutes, about 54 minutes, about 56 minutes, about 58 minutes, about 1 hour, or more. Additionally or alternatively, in some embodiments, the wound dressings are administered for about 1 hour, about 2 hour, about 3 hour, about 4 hour, about 5 hour, about 6 hour, about 7 hour, about 8 hour, about 9 hour, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more. Additionally or alternatively, in some embodiments, the wound dressings are administered for about 1 day, about 2 days, about 3 days, about 4 days, about 5 days, about 6 days, about 7 days, or more.

### Methods of Making the Wound Dressings of the Present Technology

Also disclosed herein are methods for making the wound dressings of the present technology. In one aspect, the present disclosure provides a method for making a wound dressing, providing a biopolymer containing a dye that is configured to release at least a portion of the dye in the presence of one or more proteases, an absorbent material configured to absorb the dye released by the biopolymer, a backing layer configured to provide visibility to a user of at least some of the dye absorbed by the absorbent material; and combining the biopolymer containing the dye, the absorbent material and the backing layer to make the wound dressing.

In one aspect, the present disclosure provides a method for making a wound dressing, providing a sheet of an absorbent hydrophobic foam that is configured to be in contact with the wound interface when in use, a biopolymer film containing a dye that is configured to release at least a portion of the dye in the presence of one or more proteases, a white superabsorbent pad that is configured to absorb the dye released by the biopolymer, a transparent backing film which provides visibility to a user of at least some of the dye absorbed by the white superabsorbent pad; and combining the sheet of the absorbent hydrophobic foam, the biopolymer containing the dye, the white superabsorbent material, and the transparent backing film to make the wound dressing.

In any embodiment of the method disclosed herein, the sheet of the absorbent hydrophobic foam, the biopolymer containing the dye, the white superabsorbent material, and the transparent backing film each independently comprise a wound-facing side and an environmental-facing side. Additionally or alternatively, in some embodiments of the method disclosed herein, the wound-facing side of the transparent backing film is adjoined with the environmental-facing side of the white superabsorbent material, wherein the wound-facing side of the white superabsorbent material is adjoined with the environmental-facing side of the biopolymer containing the dye, and wherein the wound-facing side of the biopolymer containing the dye is adjoined with the environmental-facing side of the sheet of the absorbent hydrophobic foam.

In any embodiment of the method disclosed herein, the sheet of the absorbent hydrophobic foam is at a thickness of about 1 mm to about 4 mm. Thus, the sheet of the absorbent hydrophobic foam is at a thickness of about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.2 mm, about 2.4 mm, about 2.6 mm, about 2.8 mm, about 3 mm, about 3.2 mm, about 3.4 mm, about 3.6 mm, about 3.8 mm, about 4 mm, or any range including and/or in between any two of the preceding values.

In any embodiment of the method disclosed herein, the biopolymer film containing a dye is placed centrally on the absorbent foam layer to form a peripheral zone on the absorbent foam layer of about 2 cm to about 4 cm. Thus, the peripheral zone that is formed on the absorbent foam layer after the biopolymer film containing the dye is placed is about 2 cm, about 2.2 cm, about 2.4 cm, about 2.6 cm, about 2.8 cm, about 3 cm, about 3.2 cm, about 3.4 cm, about 3.6 cm, about 3.8 cm, about 4 cm, or any range including and/or in between any two of the preceding values.

In any embodiment of the method disclosed herein, the biopolymer film containing the dye may further include perforations. Additionally or alternatively, in some embodiments of the method disclosed herein, the perforations in the biopolymer film containing the dye may be about 1 mm to about 10 mm. Thus, the perforations in the biopolymer film containing the dye may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

In any embodiment of the method disclosed herein, the biopolymer containing the dye may be composed of one or more of a collagen, a gelatin, an elastin, a fibronectin, or any combination thereof.

In any embodiment of the method disclosed herein, the biopolymer may comprise about 0.01 wt. % to about 10 wt. % dye. Additionally or alternatively, in some embodiments of the method disclosed herein, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values. Additionally or alternatively, in some embodiments of the method disclosed herein, the dye may be selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

In any embodiment of the method disclosed herein, the biopolymer containing the dye in the wound dressing of the present technology is configured to release at least a portion of the dye in the presence of one or more proteases in the wound. The wound dressing of the present technology is suitable for use with a variety of proteases. Typically, the proteases selected for use with the wound dressing of the present technology are associated with wound chronicity and delayed healing. Additionally or alternatively, in some embodiments of the method disclosed herein, the one or more proteases are collagenases, stromeolysins, gelatinases, elastases, fibronectinases, membrane-type MMPs, MMP-8, MMP-2 or MMP-9.

In any embodiment of the method disclosed herein, the biopolymer containing the dye may comprise at least one plasticizer. Additionally or alternatively, in some embodiments of the method disclosed herein, the biopolymer containing the dye may comprise about 0.3% w/v to about 5% w/v of at least one plasticizer. Additionally or alternatively, in some embodiments of the method disclosed herein, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values. Exemplary plasticizers include, but are not limited to, an acetylated monoglyceride, an alkyl citrate, methyl ricinoleate, glycerol, polyvinylpyrrolidone, or any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

In any embodiment of the method disclosed herein, the wound-facing side of the transparent backing film may be coated with any suitable medical grade adhesive known in the art (e.g., a polyacrylate adhesive) to secure the wound dressing to the subject while the wound dressing is configured to be in use. Additionally or alternatively, in some embodiments, of the method disclosed herein, the adhesive also secures the transparent backing film to the sheet of the absorbent hydrophobic foam, encapsulating the biopolymer containing the dye and the white superabsorbent material.

### Kits Comprising the Wound Dressings of the Present Technology

In a further related aspect, the present disclosure provides kits that include a wound dressing of any embodiment described herein and instructions for use. The kits of the present technology may also include methods for treating a wound in a subject in need thereof. The kit may optionally comprise components such as antiseptic wipes, ointment, adhesive tape, tweezers, or scissors.

### The Reduced-Pressure Wound Dressing Apparatus of the Present Technology

The present disclosure provides a reduced-pressure wound dressing apparatus is provided that includes a wound-interface layer, a biopolymer containing a dye that is configured to release at least a portion of the dye in the presence of one or more proteases, a drape comprising a pressure-sensitive adhesive in peripheral areas for sealing a wound tissue site, a canister for collecting fluids, wherein the canister is configured to be connected to the drape through a first tube connection, and a vacuum for applying negative pressure to said reduced-pressure wound dressing apparatus, wherein the vacuum is configured to be connected to the canister through a second tube connection.

In any embodiment disclosed herein, the wound-interface layer comprises an absorbent foam. Additionally or alternatively, in some embodiments, the absorbent foam of the wound-interface layer is one or more of thermoplastic elastomers, GranuFoam®, Supracor®, Grey Foam, Zotefoam, hydropolymer polyurethane foam, or any combination thereof. Additionally or alternatively, in some embodiments, the one or more thermoplastic elastomers are selected from the group consisting of styrene ethylene butylene styrene (SEBS) copolymers and thermoplastic polyurethane (TPU). Suitable absorbent foams and methods of use are described in U.S. Pat. No. 9,918,733, the entire contents of which are incorporated herein by reference.

In any embodiment disclosed herein, the wound-interface layer may be configured to be in contact with a wound when in use.

In any embodiment disclosed herein, the absorbent foam of the wound-interface layer may be mechanically or chemically compressed to increase the density of the foam at ambient pressure. A foam that is mechanically or chemically compressed may be referred to as a compressed foam, which may be characterized by a firmness factor (FF). Mechanically or chemically compressing a foam may reduce the thickness of the foam at ambient pressure, when compared to that same foam that has not be compressed. Reducing the thickness of a foam by mechanical or chemical compression may increase the density of a foam, which may increase the firmness factor (FF) of the foam. Additionally or alternatively, in some embodiments, the wound-interface layer may comprise a firmness factor (FF) of about 1 to about 5. Thus, the wound-interface layer may comprise a firmness factor (FF) of about 1, about 1.1, about 1.2, about 1.3, about 1.4, about 1.5, about 1.6, about 1.7, about 1.8, about 1.9, about 2, about 2.2, about 2.4, about 2.6, about 2.8, about 3, about 3.2, about 3.4, about 3.6, about 3.8, about 4, about 4.2, about 4.4, about 4.6, about 4.8, about 5, or any range including and/or in between any two of the preceding values.

In any embodiment disclosed herein, the wound-interface layer may comprise perforations. Additionally or alternatively, in some embodiments, the perforations of the wound-interface layer may be about 1 mm to about 10 mm in diameter. Thus, the perforations in the wound-interface layer may be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6 mm, about 6.1 mm, about 6.2 mm, about 6.3 mm, about 6.4 mm, about 6.5 mm, about 6.6 mm, about 6.7 mm, about 6.8 mm, about 6.9 mm, about 7 mm, about 7.1 mm, about 7.2 mm, about 7.3 mm, about 7.4 mm, about 7.5 mm, about 7.6 mm, about 7.7 mm, about 7.8 mm, about 7.9 mm, about 8 mm, about 8.1 mm, about 8.2 mm, about 8.3 mm, about 8.4 mm, about 8.5 mm, about 8.6 mm, about 8.7 mm, about 8.8 mm, about 8.9 mm, about 9 mm, about 9.1 mm, about 9.2 mm, about 9.3 mm, about 9.4 mm, about 9.5 mm, about 9.6 mm, about 9.7 mm, about 9.8 mm, about 9.9 mm, about 10 mm, or any range including and/or in between any two of these values.

In any embodiment disclosed herein, the wound-interface layer may comprise an antimicrobial agent. Additionally or alternatively, in some embodiments, the wound-interface layer may comprise about 0.001 wt. % to about 5 wt. % of an antimicrobial agent. Additionally or alternatively, in some embodiments the antimicrobial agent of the wound-interface layer may comprise about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.1 wt. %, about 2.2 wt. %, about 2.3 wt. %, about 2.4 wt. %, about 2.5 wt. %, about 2.6 wt. %, about 2.7 wt. %, about 2.8 wt. %, about 2.9 wt. %, about 3 wt. %, about 3.1 wt. %, about 3.2 wt. %, about 3.3 wt. %, about 3.4 wt. %, about 3.5 wt. %, about 3.6 wt. %, about 3.7 wt. %, about 3.8 wt. %, about 3.9 wt. %, about 4 wt. %, about 4.1 wt. %, about 4.2 wt. %, about 4.3 wt. %, about 4.4 wt. %, about 4.5 wt. %, about 4.6 wt. %, about 4.7 wt. %, about 4.8 wt. %, about 4.9 wt. %, about 5 wt. %, or any range including and/or in between any two of the preceding values. Additionally or alternatively, in some embodiments the antimicrobial agent of the wound-interface layer is selected from the group consisting of tetracycline, penicillins, terramycins, erythromycin, bacitracin, neomycin, polymycin B, mupirocin, clindamycin, colloidal silver, silver salts, silver sulfadiazine, chlorhexidine, povidone iodine, triclosan, sucralfate, quaternary ammonium salts, and any combination thereof.

In any embodiment disclosed herein, the biopolymer containing the dye may be composed of one or more of a collagen, a gelatin, an elastin, a fibronectin, or any combination thereof.

In any embodiment disclosed herein, the solid content of the biopolymer containing the dye may comprise about 1% w/v to about 6% w/v. Additionally or alternatively, in some embodiments, the solid content of the biopolymer containing the dye may comprise about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, about 5.1% w/v, about 5.2% w/v, about 5.3% w/v, about 5.4% w/v, about 5.5% w/v, about 5.6% w/v, about 5.7% w/v, about 5.8% w/v, about 5.9% w/v, about 6% w/v, or any range including and/or in between any two of the preceding values.

In any embodiment disclosed herein, the biopolymer may comprise about 0.01 wt. % to about 10 wt. % dye. Additionally or alternatively, in some embodiments, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values. Additionally or alternatively, in some embodiments, the dye may be selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

In any embodiment disclosed herein, the biopolymer containing the dye may be applied and dehydrated onto the wound-interface layer of the reduced-pressure wound dressing apparatus by any method known in the art. Additionally or alternatively, in some embodiments, the thickness of the biopolymer containing the dye on the wound-interface layer may be about 15 μm to about 3 mm. Additionally or alternatively, in some embodiments, the thickness of the biopolymer containing the dye on the wound-interface layer is about 15 μm, about 16 μm, about 17 μm, about 18 μm, about 19 μm, about 20 μm, about 22 μm, about 24 μm, about 26 μm, about 28 μm, about 30 μm, about 32 μm, about 34 μm, about 36 μm, about 38 μm, about 40 μm, about 42 μm, about 44 μm, about 46 μm, about 48 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, about 110 μm, about 120 μm, about 130 μm, about 140 μm, about 150 μm, about 160 μm, about 170 μm, about 180 μm, about 190 μm, about 200 μm, about 220 μm, about 240 μm, about 260 μm, about 280 μm, about 300 μm, about 320 μm, about 340 μm, about 360 μm, about 380 μm, about 400 μm, about 420 μm, about 440 μm, about 460 μm, about 480 μm, about 500 μm, about 550 μm, about 600 μm, about 650 μm, about 700 μm, about 750 μm, about 800 μm, about 850 μm, about 900 μm, about 950 μm, about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, or any range including and/or in between any two of the preceding values.

In any embodiment disclosed herein, the biopolymer containing the dye in the reduced-pressure wound dressing apparatus of the present technology is configured to release at least a portion of the dye in the presence of one or more proteases in the wound. The reduced-pressure wound dressing apparatus of the present technology is suitable for use with a variety of proteases. Typically, the proteases selected for use with the reduced-pressure wound dressing apparatus of the present technology are associated with wound chronicity and delayed healing. Additionally or alternatively, in some embodiments, the one or more proteases are collagenases, stromeolysins, gelatinases, elastases, fibronectinases, membrane-type MMPs, MMP-8, MMP-2 or MMP-9.

In any embodiment disclosed herein, the biopolymer containing the dye may comprise at least one plasticizer. Additionally or alternatively, in some embodiments, the biopolymer containing the dye may comprise about 0.3% w/v to about 5% w/v of at least one plasticizer. Additionally or alternatively, in some embodiments, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values. Exemplary plasticizers include, but are not limited to, an acetylated monoglyceride, an alkyl citrate, methyl ricinoleate, glycerol, polyvinylpyrrolidone, or any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

In any embodiment disclosed herein, the drape may be composed of a polyurethane film or an elastomeric film. Examples of an elastomeric film include, but are not limited to, natural rubber, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, ethylene vinyl acetate (EVA) film, co-polyester, or silicone. Suitable drape materials and methods of use are described in U.S. Pat. Nos. 7,534,240, 7,611,500, 9,918,733, and U.S. patent application Ser. No. 14/708,078, of which the entire contents are incorporated herein by reference.

Additionally or alternatively, in some embodiments, the thickness of the drape may be about 30 μm to about 100 μm. Thus, the thickness of the drape may be about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 52 μm, about 54 μm, about 56 μm, about 58 μm, about 60 μm, about 62 μm, about 64 μm, about 66 μm, about 68 μm, about 70 μm, about 72 μm, about 74 μm, about 76 μm, about 78 μm, about 80 μm, about 82 μm, about 84 μm, about 86 μm, about 88 μm, about 90 μm, about 92 μm, about 94 μm, about 96 μm, about 98 μm, about 100 μm, or any range including and/or in between any two of the preceding values.

In any embodiment disclosed herein, the drape may comprise a wound-facing side and an environmental-facing side.

In any embodiment disclosed herein, the drape is substantially impermeable to liquid and wound exudate. Additionally or alternatively, in some embodiments, the drape is microorganism impermeable. Additionally or alternatively, in some embodiments, the drape is semi-permeable to water vapor. In any embodiment disclosed herein, the drape may comprise a moisture vapor transmission rate (MVTR) of about 300 g/m²/24 hrs to about 20,000 g/m²/24 hrs, or about 500 g/m²/24 hrs to about 2000 g/m²/24 hrs at 37.5° C. at 50% relative humidity difference as described in ASTM E96-00. Thus, the drape may comprise a MVTR of about 300 g/m²/24 hrs, about 350 g/m²/24 hrs, about 400 g/m²/24 hrs, about 450 g/m²/24 hrs, about 500 g/m²/24 hrs, about 550 g/m²/24 hrs, about 600 g/m²/24 hrs, about 650 g/m²/24 hrs, about 700 g/m²/24 hrs, about 750 g/m²/24 hrs, about 800 g/m²/24 hrs, about 850 g/m²/24 hrs, about 900 g/m²/24 hrs, about 950 g/m²/24 hrs, about 1000 g/m²/24 hrs, about 1100 g/m²/24 hrs, about 1200 g/m²/24 hrs, about 1300 g/m²/24 hrs, about 1400 g/m²/24 hrs, about 1500 g/m²/24 hrs, about 1600 g/m²/24 hrs, about 1700 g/m²/24 hrs, about 1800 g/m²/24 hrs, about 1900 g/m²/24 hrs, about 2000 g/m²/24 hrs, about 2200 g/m²/24 hrs, about 2400 g/m²/24 hrs, about 2600 g/m²/24 hrs, about 2800 g/m²/24 hrs, about 3000 g/m²/24 hrs, about 3200 g/m²/24 hrs, about 3400 g/m²/24 hrs, about 3600 g/m²/24 hrs, about 3800 g/m²/24 hrs, about 4000 g/m²/24 hrs, about 4200 g/m²/24 hrs, about 4400 g/m²/24 hrs, about 4600 g/m²/24 hrs, about 4800 g/m²/24 hrs, about 5000 g/m²/24 hrs, about 5200 g/m²/24 hrs, about 5400 g/m²/24 hrs, about 5600 g/m²/24 hrs, about 5800 g/m²/24 hrs, about 6000 g/m²/24 hrs, about 6200 g/m²/24 hrs, about 6400 g/m²/24 hrs, about 6600 g/m²/24 hrs, about 6800 g/m²/24 hrs, about 7000 g/m²/24 hrs, about 7200 g/m²/24 hrs, about 7400 g/m²/24 hrs, about 7600 g/m²/24 hrs, about 7800 g/m²/24 hrs, about 8000 g/m²/24 hrs, about 8200 g/m²/24 hrs, about 8400 g/m²/24 hrs, about 8600 g/m²/24 hrs, about 8800 g/m²/24 hrs, about 9000 g/m²/24 hrs, about 9200 g/m²/24 hrs, about 9400 g/m²/24 hrs, about 9600 g/m²/24 hrs, about 9800 g/m²/24 hrs, about 10000 g/m²/24 hrs, about 10500 g/m²/24 hrs, about 11000 g/m²/24 hrs, about 11500 g/m²/24 hrs, about 12000 g/m²/24 hrs, about 12500 g/m²/24 hrs, about 13000 g/m²/24 hrs, about 13500 g/m²/24 hrs, about 14000 g/m²/24 hrs, about 14500 g/m²/24 hrs, about 15000 g/m²/24 hrs, about 15500 g/m²/24 hrs, about 16000 g/m²/24 hrs, about 16500 g/m²/24 hrs, about 17000 g/m²/24 hrs, about 17500 g/m²/24 hrs, about 18000 g/m²/24 hrs, about 18500 g/m²/24 hrs, about 19000 g/m²/24 hrs, about 19500 g/m²/24 hrs, about 20000 g/m²/24 hrs, or any range including and/or in between any two of the preceding values.

In any embodiment disclosed herein, the drape may comprise a pressure-sensitive adhesive in peripheral areas for sealing a wound tissue site. A “peripheral area” of the drape is an area extending inward from an external boundary (e.g., an outer edge) of the respective drape. The peripheral area of any embodiment of the drape may be an area extending inward from the external boundary about 0.1 cm to about 2.0 cm. Thus, the peripheral area may extend inward about 0.1 cm, about 0.15 cm, about 0.2 cm, about 0.25 cm, about 0.3 cm, about 0.35 cm, about 0.4 cm, about 0.45 cm, about 0.5 cm, about 0.55 cm, about 0.6 cm, about 0.65 cm, about 0.7 cm, about 0.75 cm, about 0.8 cm, about 0.85 cm, about 0.9 cm, about 0.95 cm, about 1 cm, about 1.05 cm, about 1.1 cm, about 1.15 cm, about 1.2 cm, about 1.25 cm, about 1.3 cm, about 1.35 cm, about 1.4 cm, about 1.45 cm, about 1.5 cm, about 1.55 cm, about 1.6 cm, about 1.65 cm, about 1.7 cm, about 1.75 cm, about 1.8 cm, about 1.85 cm, about 1.9 cm, about 1.95 cm, about 2.0 cm, or any range including and/or in between any two of these values.

In any embodiment disclosed herein, the first tube connection and the second tube connection may independently be a tube, pipe, hose, conduit, or any other structure with one or more lumina adapted to convey liquid between two ends. Additionally or alternatively, in some embodiments, the first tube connection and the second tube connection of the reduced-pressure wound dressing apparatus may independently be composed of polyvinyl chloride, polyethylene, polypropylene, or any combination thereof. Additionally or alternatively, in some embodiments, the canister is configured to be connected to the drape through a first tube connection. Additionally or alternatively, in some embodiments, the vacuum is configured to be connected to the canister through a second tube connection. Suitable tube connection materials and methods of use are described in U.S. Pat. Nos. 7,534,240, 7,611,500, 9,918,733, and U.S. patent application Ser. No. 14/708,078, of which the entire contents are incorporated herein by reference.

In any embodiment disclosed herein, the reduced-pressure wound dressing apparatus comprises a vacuum for applying negative pressure to the tube connections. Additionally or alternatively, in some embodiments, negative pressure refers to a pressure less than local ambient pressure, such as the pressure in a local environment external to a sealed wound site. Additionally or alternatively, in some embodiments, the vacuum for applying negative pressure may be a vacuum pump, a suction pump, a micro-pump, or a wall vacuum port available in many healthcare facilities. Additionally or alternatively, in some embodiments, the vacuum is used to apply negative pressure to a wound. Additionally or alternatively, in some embodiments, the negative pressure applied to a wound may be about −5 mm Hg to about −500 mm Hg, or about −75 mm Hg to about −300 mm Hg. Thus, the negative pressure applied to a wound may be about −5 mm Hg, about −6 mm Hg, about −7 mm Hg, about −8 mm Hg, about −9 mm Hg, about −10 mm Hg, about −11 mm Hg, about −12 mm Hg, about −13 mm Hg, about −14 mm Hg, about −15 mm Hg, about −16 mm Hg, about −17 mm Hg, about −18 mm Hg, about −19 mm Hg, about −20 mm Hg, about −22 mm Hg, about −24 mm Hg, about −26 mm Hg, about −28 mm Hg, about −30 mm Hg, about −32 mm Hg, about −34 mm Hg, about −36 mm Hg, about −38 mm Hg, about −40 mm Hg, about −42 mm Hg, about −44 mm Hg, about −46 mm Hg, about −48 mm Hg, about −50 mm Hg, about −52 mm Hg, about −54 mm Hg, about −56 mm Hg, about −58 mm Hg, about −60 mm Hg, about −62 mm Hg, about −64 mm Hg, about −66 mm Hg, about −68 mm Hg, about −70 mm Hg, about −72 mm Hg, about −74 mm Hg, about −76 mm Hg, about −78 mm Hg, about −80 mm Hg, about −82 mm Hg, about −84 mm Hg, about −86 mm Hg, about −88 mm Hg, about −90 mm Hg, about −92 mm Hg, about −94 mm Hg, about −96 mm Hg, about −98 mm Hg, about −100 mm Hg, about −110 mm Hg, about −120 mm Hg, about −130 mm Hg, about −140 mm Hg, about −150 mm Hg, about −160 mm Hg, about −170 mm Hg, about −180 mm Hg, about −190 mm Hg, about −200 mm Hg, about −220 mm Hg, about −240 mm Hg, about −260 mm Hg, about −280 mm Hg, about −300 mm Hg, about −320 mm Hg, about −340 mm Hg, about −360 mm Hg, about −380 mm Hg, about −400 mm Hg, about −420 mm Hg, about −440 mm Hg, about −460 mm Hg, about −480 mm Hg, about −500 mm Hg, or any range including and/or in between any two of these values. Methods of use of negative pressure therapy devices are described in U.S. Pat. Nos. 7,534,240, 7,611,500, 9,918,733, and U.S. patent application Ser. No. 14/708,078, of which the entire contents are incorporated herein by reference.

In one aspect, the present disclosure provides a method for detecting protease activity levels in a wound in a subject in need thereof, wherein the method comprises contacting the wound with the wound-interface layer of the reduced-pressure wound dressing apparatus of any embodiment described herein, applying negative pressure to the wound using the vacuum of the reduced-pressure wound dressing apparatus, collecting wound exudate via the first tube connection and/or canister of the reduced-pressure wound dressing apparatus, and detecting a colorimetric signal in the collected wound exudate, wherein detection of the colorimetric signal indicates protease activity in the wound. Additionally or alternatively, in some embodiments, the wound may be an acute wound or a chronic wound. Additionally or alternatively, in some embodiments, the wound is an acute wound selected from the group consisting of surgical wounds, trauma wounds, burns, graft sites, and donor sites. Additionally or alternatively, the wound is a chronic wound selected from the group consisting of infectious wounds, venous ulcers, arterial ulcers, ischemic ulcers, decubitis ulcers, and diabetic ulcers.

Any method known to those in the art for administering a reduced-pressure wound dressing apparatus to an acute wound or a chronic wound disclosed herein may be employed. Suitable methods include in vitro or in vivo methods. In vivo methods typically include the administration of one or more reduced-pressure wound dressing apparatuses to a subject in need thereof, suitably a human.

In another aspect, the present disclosure provides a method for detecting delays in wound healing in a subject in need thereof, wherein the method comprises contacting the wound with the wound-interface layer of the reduced-pressure wound dressing apparatus of any embodiment described herein, applying negative pressure via the vacuum of the reduced-pressure wound dressing apparatus, collecting wound exudate via the first tube connection and/or canister of the reduced-pressure wound dressing apparatus, and detecting a first colorimetric signal in the wound exudate at a first time period, detecting a second colorimetric signal in the wound exudate at a second time period, and detecting a delay in wound healing when the second colorimetric signal is greater than the first colorimetric signal.

In any embodiment of the methods of the present technology, a reduced-pressure wound dressing apparatus disclosed herein is administered to a subject in need thereof. Without wishing to be bound by theory, it is believed that, the wound exudate of the subject may vary in viscosity and quantity, thus affecting the appropriate temporal window for administering the reduced-pressure wound dressing apparatuses disclosed herein. Additionally or alternatively, in some embodiments, the diffusion rate of the wound exudate may vary depending on the structure of the reduced-pressure wound dressing apparatus disclosed herein. Additionally or alternatively, in some embodiments, the reduced-pressure wound dressing apparatuses are administered for about 1 minute or more. Additionally or alternatively, in some embodiments, the reduced-pressure wound dressing apparatuses are administered for about 1 minute, about 2 minutes, about 3 minutes, about 4 minutes, about 5 minutes, about 6 minutes, about 7 minutes, about 8 minutes, about 9 minutes, about 10 minutes, or more. Additionally or alternatively, in some embodiments, the reduced-pressure wound dressing apparatuses are administered for about 10 minutes, about 11 minutes, about 12 minutes, about 13 minutes, about 14 minutes, about 15 minutes, about 16 minutes, about 17 minutes, about 18 minutes, about 19 minutes, about 20 minutes, or more. Additionally or alternatively, in some embodiments, the reduced-pressure wound dressing apparatuses are administered for about 20 minutes, about 22 minutes, about 24 minutes, about 26 minutes, about 28 minutes, about 30 minutes, about 32 minutes, about 34 minutes, about 36 minutes, about 38 minutes, about 40 minutes, about 42 minutes, about 44 minutes, about 46 minutes, about 48 minutes, about 50 minutes, about 52 minutes, about 54 minutes, about 56 minutes, about 58 minutes, about 1 hour, or more. Additionally or alternatively, in some embodiments, the reduced-pressure wound dressing apparatuses are administered for about 1 hour, about 2 hour, about 3 hour, about 4 hour, about 5 hour, about 6 hour, about 7 hour, about 8 hour, about 9 hour, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, or more.

Also disclosed herein are methods for making the reduced-pressure wound dressing apparatuses of the present technology. In one aspect, the present disclosure provides a method for making a reduced-pressure wound dressing apparatus, providing a biopolymer containing a dye and configured to release at least a portion of the dye when in the presence of one or more proteases, a drape comprising a pressure-sensitive adhesive in peripheral areas for sealing a wound tissue site, a canister for collecting fluids, wherein the canister is configured to be connected to the drape through a first tube connection, a vacuum for applying negative pressure to said reduced-pressure wound dressing apparatus, wherein the vacuum is configured to be connected to the canister through a second tube connection, and combining the biopolymer containing the dye, the drape, the canister, and the vacuum to make the reduced-pressure wound dressing apparatus.

In one aspect, the present disclosure provides a method for making a reduced-pressure wound dressing apparatus, providing an open cell foam that is configured to be in contact with the wound interface when in use, a biopolymer containing a dye and configured to release at least a portion of the dye when in the presence of one or more proteases, a drape comprising a pressure-sensitive adhesive in peripheral areas for sealing a wound tissue site, a canister for collecting fluids, wherein the canister is configured to be connected to the drape through a first tube connection, a vacuum for applying negative pressure to said reduced-pressure wound dressing apparatus, wherein the vacuum is configured to be connected to the canister through a second tube connection, and combining the biopolymer containing the dye, the drape, the canister, and the vacuum to make the reduced-pressure wound dressing apparatus.

In any embodiment of the method disclosed herein, the biopolymer containing the dye may be applied and dehydrated onto the wound-interface layer of the reduced-pressure wound dressing apparatus by any method known in the art.

In any embodiment of the method disclosed herein, the biopolymer containing the dye may be composed of one or more of a collagen, a gelatin, an elastin, a fibronectin, or any combination thereof.

In any embodiment of the method disclosed herein, the biopolymer may comprise about 0.01 wt. % to about 10 wt. % dye. Additionally or alternatively, in some embodiments of the method disclosed herein, the biopolymer may contain about 0.01 wt. %, about 0.05 wt. %, about 0.1 wt. %, about 0.15 wt. %, about 0.2 wt. %, about 0.25 wt. %, about 0.3 wt. %, about 0.35 wt. %, about 0.4 wt. %, about 0.45 wt. %, about 0.5 wt. %, about 0.55 wt. %, about 0.6 wt. %, about 0.65 wt. %, about 0.7 wt. %, about 0.75 wt. %, about 0.8 wt. %, about 0.85 wt. %, about 0.9 wt. %, about 0.95 wt. %, about 1 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2 wt. %, about 2.2 wt. %, about 2.4 wt. %, about 2.6 wt. %, about 2.8 wt. %, about 3 wt. %, about 3.2 wt. %, about 3.4 wt. %, about 3.6 wt. %, about 3.8 wt. %, about 4 wt. %, about 4.2 wt. %, about 4.4 wt. %, about 4.6 wt. %, about 4.8 wt. %, about 5 wt. %, about 5.2 wt. %, about 5.4 wt. %, about 5.6 wt. %, about 5.8 wt. %, about 6 wt. %, about 6.2 wt. %, about 6.4 wt. %, about 6.6 wt. %, about 6.8 wt. %, about 7 wt. %, about 7.2 wt. %, about 7.4 wt. %, about 7.6 wt. %, about 7.8 wt. %, about 8 wt. %, about 8.2 wt. %, about 8.4 wt. %, about 8.6 wt. %, about 8.8 wt. %, about 9 wt. %, about 9.2 wt. %, about 9.4 wt. %, about 9.6 wt. %, about 9.8 wt. %, about 10 wt. %, or any range including and/or in between any two of these values. Additionally or alternatively, in some embodiments of the method disclosed herein, the dye may be selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, sudan III, methyl green, oil red, pyronin Y, purpurin, quantum dots, phloxine B, picric acid, carbon nanotubes, fuchsins, resazurin, trichromes, food coloring, tattoo ink, and any combination thereof.

In any embodiment of the method disclosed herein, the biopolymer containing the dye in the wound dressing of the present technology is configured to release at least a portion of the dye in the presence of one or more proteases in the wound. The reduced-pressure wound dressing apparatus of the present technology is suitable for use with a variety of proteases. Typically, the proteases selected for use with the reduced-pressure wound dressing apparatus of the present technology are associated with wound chronicity and delayed healing. Additionally or alternatively, in some embodiments of the method disclosed herein, the one or more proteases are collagenases, stromeolysins, gelatinases, elastases, fibronectinases, membrane-type MMPs, MMP-8, MMP-2 or MMP-9.

In any embodiment of the method disclosed herein, the biopolymer containing the dye may comprise at least one plasticizer. Additionally or alternatively, in some embodiments of the method disclosed herein, the biopolymer containing the dye may comprise about 0.3% w/v to about 5% w/v of at least one plasticizer. Additionally or alternatively, in some embodiments of the method disclosed herein, the at least one plasticizer of the biopolymer containing the dye may comprise about 0.3% w/v, about 0.32% w/v, about 0.34% w/v, about 0.36% w/v, about 0.38% w/v, about 0.4% w/v, about 0.42% w/v, about 0.44% w/v, about 0.46% w/v, about 0.48% w/v, about 0.5% w/v, about 0.52% w/v, about 0.54% w/v, about 0.56% w/v, about 0.58% w/v, about 0.6% w/v, about 0.62% w/v, about 0.64% w/v, about 0.66% w/v, about 0.68% w/v, about 0.7% w/v, about 0.72% w/v, about 0.74% w/v, about 0.76% w/v, about 0.78% w/v, about 0.8% w/v, about 0.82% w/v, about 0.84% w/v, about 0.86% w/v, about 0.88% w/v, about 0.9% w/v, about 0.92% w/v, about 0.94% w/v, about 0.96% w/v, about 0.98% w/v, about 1% w/v, about 1.1% w/v, about 1.2% w/v, about 1.3% w/v, about 1.4% w/v, about 1.5% w/v, about 1.6% w/v, about 1.7% w/v, about 1.8% w/v, about 1.9% w/v, about 2% w/v, about 2.1% w/v, about 2.2% w/v, about 2.3% w/v, about 2.4% w/v, about 2.5% w/v, about 2.6% w/v, about 2.7% w/v, about 2.8% w/v, about 2.9% w/v, about 3% w/v, about 3.1% w/v, about 3.2% w/v, about 3.3% w/v, about 3.4% w/v, about 3.5% w/v, about 3.6% w/v, about 3.7% w/v, about 3.8% w/v, about 3.9% w/v, about 4% w/v, about 4.1% w/v, about 4.2% w/v, about 4.3% w/v, about 4.4% w/v, about 4.5% w/v, about 4.6% w/v, about 4.7% w/v, about 4.8% w/v, about 4.9% w/v, about 5% w/v, or any range including and/or in between any two of the preceding values. Exemplary plasticizers include, but are not limited to, an acetylated monoglyceride, an alkyl citrate, methyl ricinoleate, glycerol, polyvinylpyrrolidone, or any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

In a further related aspect, the present disclosure provides kits that include a reduced-pressure wound dressing apparatus of any embodiment described herein and instructions for use. The kits of the present technology may also include methods for treating a wound in a subject in need thereof. The kits may further comprise additional canisters, drapes, medical-grade adhesive, or spare tubing. The kits may optionally comprise components such as antiseptic wipes, ointment, adhesive tape, tweezers, or scissors.

### EXAMPLES

The present technology is further illustrated by the following examples, which should not be construed as limiting in any way.

### Example 1: Detecting the Release of Dye from the Biopolymer

To determine the ability of the biopolymer containing the dye to release at least a portion of the dye in the presence of a protease, samples of the biopolymer containing the dye will be incubated in solutions containing collagenase (approximately 238 units/110 μL) or no collagenase (110 μL total volume, control). The color of the solution will monitored with respect to time by ultraviolet-visible spectroscopy (UV-Vis). The dye released into the solution will be reflective of the enzymatic activity of the collagenase in solution, and thus will be representative of protease activity present in wound exudate. Accordingly, it is anticipated that the administration of the wound dressings of the present technology will result in the release of the layer containing the biopolymer and the dye, and in the detection of protease activity in the wound, an indication of delayed wound healing.

These results will demonstrate that the wound dressings of the present technology are useful for detecting protease activity levels in a wound in a subject in need thereof.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A wound dressing comprising:
a biopolymer containing a dye and configured to release at least a portion of the dye in the presence of one or more proteases;
an absorbent material configured to absorb the dye released by the biopolymer;
a backing layer configured to provide visualization of at least some of the dye
absorbed by the absorbent material as a result of protease-mediated degradation of the biopolymer,
optionally wherein
the thickness of the biopolymer is about 15 pm to about 3 mm, and/or wherein the solid content of the biopolymer comprises about 1% w/v to about 6% w/v, or
the thickness of the absorbent material is about 15 pm to about 500 pm, or
the thickness of the backing layer is about 10 pm to about 1000 pm, or about 30 pm to about 60 pm.

2. The wound dressing of claim 1, wherein the biopolymer
is in the form of a film, optionally wherein the film containing the biopolymer and the dye comprises perforations, wherein the perforations are about 1 mm to about 10 mm in diameter, or
is composed of one or more of a collagen, a gelatin, an elastin, a fibronectin, or any combination thereof, or
comprises about 0.01 wt. % to about 10 wt. % dye.

3. (canceled)

4. (canceled)

5. (canceled)

6. (canceled)

7. (canceled)

8. The wound dressing of claim 1, wherein the dye is selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, Sudan III, methyl green, oil red, pyronin Y, purpurin, quantum dots, phloxine B, picric acid, carbon nanotubes, fuchsins, resazurin, trichromes, food coloring, tattoo ink, and any combination thereof, or
wherein the one or more proteases are collagenases, stromeolysins, gelatinases, elastases, fibronectinases, membrane-type MMPs, MMP-8, MMP-2 or MMP-9.

9. (canceled)

10. (canceled)

11. The wound dressing of claim 1, wherein the biopolymer comprises at least one plasticizer, optionally wherein the biopolymer comprises about 0.3% w/v to about 5% w/v of at least one plasticizer, wherein the at least one plasticizer is selected from the group consisting of an acetylated monoglyceride, an alkyl citrate, methyl ricinoleate, glycerol, polyvinylpyrrolidone, and any combination thereof, and wherein the alkyl citrate is triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof.

12. (canceled)

13. (canceled)

14. (canceled)

15. The wound dressing of claim 1, wherein the absorbent material is substantially white or a superabsorbent, optionally wherein the superabsorbent of the absorbent material comprises about 5 wt. % to about 60 wt. % or comprises sodium polyacrylate.

16. (canceled)

17. (canceled)

18. (canceled)

19. (canceled)

20. The wound dressing of claim 1, wherein the backing layer
is transparent or semi-transparent, or
is selected from the group consisting of polyurethane, polyalkoxy alkyl acrylate, polyalkoxy alkyl methacrylates, and any combination thereof, or
comprises a moisture vapor transmission rate (MVTR) of about 300 g/m²/24 hrs to about 20,000 g/m²/24 hrs, or about 500 g/m²/24 hrs to about 2000 g/m²/24 hrs.

21. (canceled)

22. (canceled)

23. (canceled)

24. The wound dressing of claim 1, further comprising a wound-interface layer, optionally wherein
the thickness of the wound-interface layer is about 15 μm to about 500 μm, or
the wound-interface layer comprises about 0.001 wt. % to about 5 wt. % of an antimicrobial agent, optionally wherein the antimicrobial agent is selected from the group consisting of tetracycline, penicillins, terramycins, erythromycin, bacitracin, neomycin, polymycin B, mupirocin, clindamycin, colloidal silver, silver salts, silver sulfadiazine, polyhexanide, chlorhexidine, povidone iodine, triclosan, sucralfate, quaternary ammonium salts, and any combination thereof, or
the wound-interface layer is an absorbent foam, optionally wherein the absorbent foam of the wound-interface layer is one or more of thermoplastic elastomers, GranuFoam®, Supracor®, Grey Foam, Zotefoam, hydropolymer polyurethane foam, or any combination thereof, optionally wherein the thermoplastic elastomers are selected from the group consisting of styrene ethylene butylene styrene (SEBS) copolymers and thermoplastic polyurethane (TPU), or
the wound-interface layer comprises perforations, optionally wherein the perforations are about 1 mm to about 10 mm in diameter.

25. (canceled)

26. (canceled)

27. (canceled)

28. (canceled)

29. (canceled)

30. (canceled)

31. (canceled)

32. (canceled)

33. A reduced-pressure wound dressing apparatus comprising:
a wound-interface layer;
a biopolymer containing a dye and configured to release at least a portion of the dye in the presence of one or more proteases;
a drape comprising a pressure-sensitive adhesive in peripheral areas for sealing a wound tissue site;
a canister for collecting fluids, wherein the canister is configured to be connected to the drape through a first tube connection; and
a vacuum for applying negative pressure to said reduced-pressure wound dressing apparatus, wherein the vacuum is configured to be connected to the canister through a second tube connection,
optionally wherein
the wound-interface layer comprises a firmness factor (FF) of about 1 to about 5, or
the thickness of the biopolymer is about 15 pm to about 3 mm, and/or wherein the solid content of the biopolymer comprises about 1% w/v to about 6% w/v, or
the thickness of the drape is about 30 pm to about 100 pm, or
the first tube connection and the second tube connection may independently be selected from the group consisting of polyvinyl chloride, polyethylene, polypropylene, and any combination thereof, or
the vacuum is used to apply negative pressure to a wound, optionally wherein the negative pressure applied to a wound may be about −5 mm Hg to about −500 mm Hg, or about −75 mm Hg to about −300 mm Hg.

34. The reduced-pressure wound dressing apparatus of claim 33, wherein the wound-interface layer
is an absorbent foam, optionally wherein the absorbent foam of the wound-interface layer is one or more of thermoplastic elastomers, GranuFoam®, Supracor®, Grey Foam, Zotefoam, hydropolymer polyurethane foam, or any combination thereof, optionally wherein the thermoplastic elastomers are selected from the group consisting of styrene ethylene butylene styrene (SEBS) copolymers and thermoplastic polyurethane (TPU), or
comprises perforations, optionally wherein the perforations are about 1 mm to about 10 mm in diameter, or
comprises about 0.001 wt. % to about 5 wt. % of an antimicrobial agent, optionally wherein the antimicrobial agent is selected from the group consisting of tetracycline, penicillins, terramycins, erythromycin, bacitracin, neomycin, polymycin B, mupirocin, clindamycin, colloidal silver, silver salts, silver sulfadiazine, polyhexanide, chlorhexidine, povidone iodine, triclosan, sucralfate, quaternary ammonium salts, and any combination thereof.

35. (canceled)

36. (canceled)

37. (canceled)

38. (canceled)

39. (canceled)

40. (canceled)

41. (canceled)

42. The reduced-pressure wound dressing apparatus of claim 33, wherein the biopolymer
is composed of one or more of a collagen, a gelatin, an elastin, a fibronectin, or any combination thereof,
comprises about 0.01 wt. % to about 10 wt. % dye, or
comprises at least one plasticizer, optionally wherein the biopolymer may comprise about 0.3% w/v to about 5% w/v of the at least one plasticizer, or wherein the at least one plasticizer is selected from the group consisting of an acetylated monoglyceride, an alkyl citrate, methyl ricinoleate, glycerol, polyvinylpyrrolidone, and any combination thereof, and wherein the alkyl citrate is triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, or any combination thereof, or
is applied and dehydrated onto the wound-interface layer.

43. (canceled)

44. The reduced-pressure wound dressing apparatus of claim 33, wherein the dye is selected from the group consisting of direct red 80, bromophenol blue, toluidine blue, fluorescein isothiocyanate (FITC), 4′,6-diamidino-2-phenylindole (DAPI), methylene blue, erythrosine B, ponceau S, alura red, SYBR green, alcian blue, brilliant blue G, calcein blue, cardio green, crystal violet, nile blue, fluoroMax, india ink, brilliant blue, indigo carmine, Sudan III, methyl green, oil red, pyronin Y, purpurin, quantum dots, phloxine B, picric acid, carbon nanotubes, fuchsins, resazurin, trichromes, food coloring, tattoo ink, and any combination thereof, or
wherein the one or more proteases are collagenases, stromeolysins, gelatinases, elastases, fibronectinases, membrane-type MMPs, MMP-8, MMP-2 or MMP-9.

45. (canceled)

46. (canceled)

47. (canceled)

48. (canceled)

49. (canceled)

50. (canceled)

51. (canceled)

52. (canceled)

53. The reduced-pressure wound dressing apparatus of claim 33, wherein the drape
comprises a polyurethane film or an elastomeric film, optionally wherein the elastomeric film is natural rubber, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, ethylene vinyl acetate (EVA) film, co-polyester, silicone, or any combination thereof, or
comprises a moisture vapor transmission rate (MVTR) of about 300 g/m²/24 hrs to about 20,000 g/m²/24 hrs, or about 500 g/m²/24 hrs to about 2000 g/m²/24 hrs.

54. (canceled)

55. (canceled)

56. (canceled)

57. (canceled)

58. (canceled)

59. (canceled)

60. A method for detecting protease activity levels in a wound in a subject in need thereof, comprising:
a. administering to the wound a wound dressing of claim 1; and
b. detecting a colorimetric signal in the absorbent material of the wound dressing, wherein the presence of the colorimetric signal indicates protease activity in the wound.

61. A method for detecting protease activity levels in a wound in a subject in need thereof, comprising:
a. contacting the wound with the wound-interface layer of the reduced-pressure wound dressing apparatus of claim 33;
b. applying negative pressure using the vacuum of the reduced-pressure wound dressing apparatus;
c. collecting wound exudate via the first tube connection and/or canister of the reduced-pressure wound dressing apparatus; and
d. detecting a colorimetric signal in the collected wound exudate, wherein detection of the colorimetric signal indicates protease activity in the wound.

62. A method for detecting delays in wound healing in a subject in need thereof, comprising:
a. administering to the wound a wound dressing of claim 1;
b. determining a first protease activity level by detecting a first colorimetric signal in the absorbent material of the wound dressing when the wound dressing is administered to the subject; and
c. determining a second protease activity level by detecting a second colorimetric signal in the absorbent material of the wound dressing about 10 minutes to about 7 days after the wound dressing is administered to the subject;
wherein wound healing is delayed when the second protease activity level is greater compared to the first protease activity level.

63. A method for detecting delays in wound healing in a subject in need thereof, comprising:
a. administering to the wound a wound dressing of claim 1;
b. detecting a colorimetric signal in the absorbent material of the wound dressing, wherein the colorimetric change indicates elevated protease activity levels; and
c. determining a protease activity level compared to a pre-determined reference level.

64. A method for detecting delays in wound healing in a subject in need thereof, comprising:
a. contacting the wound with the wound-interface layer of the reduced- pressure wound dressing apparatus of claim 33;
b. applying negative pressure via the vacuum of the reduced-pressure wound dressing apparatus;
c. collecting wound exudate via the first tube connection and/or canister of the reduced-pressure wound dressing apparatus; and
d. detecting a first colorimetric signal in the wound exudate at a first time period;
e. detecting a second colorimetric signal in the wound exudate at a second time period; and
f. detecting a delay in wound healing when the second colorimetric signal is greater than the first colorimetric signal.

65. (canceled)

66. A method for making a reduced-pressure wound dressing apparatus:
a. providing:
a biopolymer containing a dye that is configured to release at least a portion of the dye when in the presence of one or more proteases;
a drape comprising a pressure-sensitive adhesive in peripheral areas for sealing a wound tissue site;
a canister for collecting fluids, wherein the canister is configured to be connected to the drape through a first tube connection;
a vacuum for applying negative pressure to said reduced-pressure wound dressing apparatus, wherein the vacuum is configured to be connected to the canister through a second tube connection; and
b. combining the biopolymer containing the dye, the drape, the canister, and the vacuum to make the reduced-pressure wound dressing apparatus.

67. A kit comprising the wound dressing of claim 1, and instructions for use.

68. A kit comprising the reduced-pressure wound dressing apparatus of claim 33, and instructions for use.
